(19) United States
(12) Patent Application Publication
(10) Pub. No.: US 2022/0257298 A1
(43) Pub. Date: Aug. 18, 2022

(54) Devices and Methods for Treating Ear, Nose, and Throat Afflictions

(71) Applicant: Arrinex, Inc., Redwood City, CA (US)

(72) Inventors: William Jason Fox, San Mateo, CA (US); Vahid Saadat, Atherton, CA (US); David Moosavi, Redwood City, CA (US); Sherwin Llamido, Union City, CA (US); Roman Turovskiy, San Francisco, CA (US); William Gould, Santa Fe, NM (US); Matt Allison Herron, Hayward, CA (US)

(21) Appl. No.: 17/623,036

(22) PCT Filed: Jul. 8, 2020
(86) PCT No.: PCT/US2020/041248

§ 371 Date: Dec. 27, 2021

Related U.S. Application Data
(60) Provisional application No. 62/872,195, filed on Jul. 9, 2019.

(51) Int. Cl. A61B 18/02

(52) U.S. Cl. CPC A61B 18/02; A61B 2018/00327

(57) ABSTRACT

Devices and methods for treating conditions such as rhinitis are disclosed herein where a distal end of a probe shaft is introduced through the nasal cavity where the distal end has an end effector with a first configuration having a low-profile which is shaped to manipulate tissue within the nasal cavity. The distal end may be positioned into proximity of a nasal tissue region having at least one nasal nerve. Once suitably positioned, the distal end may be reconfigured from the first configuration to a second configuration which is shaped to contact and follow the nasal tissue region and the at least one nasal nerve may then be ablated via the distal end. Ablation may be performed using various mechanisms, such as cryotherapy, and optionally under direct visualization.

### CROSS-REFERENCE TO RELATED APPLICATIONS

The present application claims the benefit of U.S. Provisional Application No. 62/872,195 filed on Jul. 9, 2019, the contents of which is hereby incorporated by reference in its entirety.

### FIELD

The present disclosure is related to devices and methods for treating regions of tissue. More particularly, the present disclosure is related to devices and methods for treating regions of tissue such as through cryotherapies including hypothermic cooling and cryogenic ablation for treating ear, nose, and throat (ENT) afflictions such as rhinitis.

### BACKGROUND

Unless otherwise indicated herein, the materials described in this section are not prior art to the claims in this application and are not admitted to be prior art by inclusion in this section.

The human nose is responsible for warming, humidifying, and filtering inspired air. The nose is mainly formed of cartilage, bone, mucous membranes, and skin. The right and left nasal cavities extend posteriorly to the soft palate, where they merge to form the posterior choanae. The posterior choanae opens into the nasopharynx. The roof of the nose is formed, in part, by a bone known as the cribriform plate. The cribriform plate contains numerous tiny perforations through which sensory nerve fibers extend to the olfactory bulbs. The sensation for smell occurs when inhaled odors contact a small area of mucosa in the superior region of the nose, stimulating the nerve fibers that lead to the olfactory bulbs.

The nasal turbinates are three bony processes that extend medially from the lateral walls of the nose and are covered with mucosal tissue. These turbinates serve to increase the interior surface area of the nose and to impart warmth and moisture to air that is inhaled through the nose. The mucosal tissue that covers the turbinates is capable of becoming engorged with blood and swelling, or becoming substantially devoid of blood and shrinking, in response to changes in physiologic or environmental conditions. The curved edge of each turbinate defines a passage way known as a meatus. For example, the inferior meatus is a passageway that passes beneath the inferior turbinate. Ducts, known as the nasolacrimal ducts, drain tears from the eyes into the nose through openings located within the inferior meatus. The middle meatus is a passageway that is lateral to the middle turbinate, inferior to its attachment to the lateral wall. The middle meatus contains the semilunar hiatus, with openings or ostia leading into the maxillary, frontal, and anterior ethmoid sinuses. The superior meatus is located between the superior and middle turbinates.

The turbinates are autonomic ally innervated by nerves arising from the vidian nerve. The vidian nerve contains sympathetic and parasympathetic afferents that can modulate the function of the soft tissue covering the turbinates to either increase (parasympathetic) or decrease (sympathetic) the activity of the submucosal layer. The vidian nerve travels to the sphenopalatine ganglion via the pterygoid canal. Some of the fibers from the sphenopalatine ganglion (SPG) enter the nasal cavity through the sphenopalatine foramen (SPF). Exclusive of the SPF, additional posterolateral neurovascular rami project from the SPG to supply the nasal mucosa. The most common locations for these rami are within 1 cm posterosuperior to the horizontal attachment of the inferior turbinate, within 5 mm anteroinferior to this attachment, and proximate to the palatine bone via a foramen distinct from the SPF. Interfascicle anastomotic loops are, in some cases, associated with at least three accessory nerves. Each accessory nerve could be traced directly to the SPG or the greater palatine nerve.

Rhinitis is defined as inflammation of the membranes lining the nose, characterized by nasal symptoms including itching, rhinorrhea, and/or nasal congestion. Chronic rhinitis affects millions of people and is a leading cause for patients to seek medical care. Medical treatment has been shown to have limited effects for chronic rhinitis sufferers and requires daily medication use or onerous allergy treatments, and up to 20% of patients may be refractory.

In addition to the existing medications, turbinate reduction surgery (e.g., radiofrequency-based and micro-debridement-based surgeries) has been shown to have a temporary duration of effect of 1-2 years, and can result in complications including mucosal sloughing, acute pain and swelling, overtreatment, and bone damage. Additionally, turbinate reduction surgery does not treat the symptom of rhinorrhea.

It is thought that parasympathetic effect of the vidian nerve predominately controls autonomic balance, and accordingly transecting it may result in decreased rhinitis and congestion. This pathophysiology has been confirmed as surgical treatment of the vidian nerve has indeed shown a reduction in some rhinitis symptoms; however, the procedure is invasive, time consuming, and potentially can result in chronic dry eyes because the autonomic fibers in the vidian nerve also supply the lacrimal glands.

Thermal therapies may represent a solution to the above limitations of prior treatments of ENT afflictions such as rhinitis. This class of therapies treats tissues by inducing temperature changes that selectively create tissue alterations, sometimes causing temporary or permanent damage. Depending on the type of tissue and the region of the body targeted for treatment, the application of thermal energy may provide various benefits, including treatment of cardiac arrhythmia, destruction of cancerous tissue masses, and alteration of nerve signaling pathways. Tissue ablation refers to a class of thermal therapies that causes destructive tissue damage. This damage may be induced via the application of heat (for example, with radiofrequency, laser, microwave, high intensity focused ultrasound (HIFU), or resistive heating methods) or via the application of cooling energy (for example, using cryoablation methods).

The term “cryotherapy” describes a class of thermal therapies that involve inducing cool or cold temperatures in body tissues, and includes the therapies generally referred to as therapeutic hypothermia and cryoablation. Depending on the temperatures and exposure times involved, the clinical goals of various cryotherapies may range from improved tissue healing/recovery (for example, as with therapeutic hypothermia employed during physical therapy sessions) to selective tissue damage or destruction (for example, during cryoablation used for neuromodulation or tumor-destruction purposes). Any tissue damage introduced during cryotherapy may be temporary or permanent, depending on the tissues treated and the characteristics of the therapy delivered.

Various cryotherapy techniques have recently been gaining in popularity for use in ENT procedures. Applications include treatments for rhinitis, enlarged turbinates, and other clinical pathologies. Modern cryotherapy for ENT is often delivered by using a compressed cryogen liquid (such as nitrous oxide) that provides a source of cooling as it expands into a gas during a transition to atmospheric pressure. This method for delivering a cold therapy eliminates the need for the complicated systems that are generally associated with thermoelectric/Peltier effect cooling and circulating fluid-based cooling, for example the need for pumps, wires, and/or other electrical hardware.

Accompanying the recent surge in popularity of cryotherapy for ENT applications, the devices, systems, and methods for delivery of cryotherapy for ENT have evolved and improved as well. Some advances in equipment and technique are geared towards improvements in medical outcomes, while others are related to either business or practical objectives. For example, ENT procedures are increasingly being delivered in outpatient office-based settings, and equipment and techniques utilized in this milieu may differ considerably from what is considered practical and safe for use within a hospital. However, even with these recent technological advances, some limitations remain with existing state-of-the-art cryotherapy equipment.

As such, the field of cryotherapy for ENT applications would be meaningfully improved if existing limitations known to those who are skilled in the art, were addressed with practical and cost-efficient solutions. Continuing to improve cryotherapy and other thermal therapy devices and techniques would enable more physicians to carry out procedures, more patients to receive procedures, and for patients who receive procedures to experience better outcomes.

### SUMMARY

The present disclosure is related to systems, devices, and methods for delivering cryotherapy interventions. More specifically, the present disclosure relates to delivering cryotherapy interventions for ENT afflictions. The present disclosure can be particularly useful when treating patients during office-based procedures, or in other situations where general anesthesia is not available, practical, and/or advisable. The present disclosure can be particularly useful during cryotherapy procedures applied within the upper airway.

The present disclosure provides methods, devices, and systems that advance the delivery of cryotherapy with solutions that improve the balance between simplicity, practicality, and effectiveness. More specifically, the systems, the devices, and/or the methods of the present disclosure allow for cryotherapy to be delivered in an improved way in the nasal cavity or other body lumens. Accomplishing this is valuable because it will improve the patient experience when receiving these important treatments which may encourage more patients to elect to receive said treatments.

In one example, the present disclosure provides a device. The device includes a probe shaft having a distal end and a proximal end. The probe shaft has a curved portion such that a longitudinal axis of a distal portion of the probe shaft has a non-zero angle with respect to a longitudinal axis of a proximal portion of the probe shaft. A flexibility of the proximal portion of the probe shaft is greater than a flexibility of the distal portion of the probe shaft. The device also includes a housing coupled to the proximal end of the probe shaft, and a handle coupled to the housing. The device also includes an end effector coupled to the distal end of the probe shaft. The end effector defines an atraumatic surface when the distal end of the probe shaft is advanced through a nasal cavity of a patient and is positioned proximate to a nasal tissue region having at least one nasal nerve, and the end effector is configured to transmit lateral pressure against the nasal tissue region. The device also includes a trigger positioned in the handle. Activation of the trigger causes the end effector to ablate the at least one nasal nerve when the end effector is in contact against the nasal tissue region.

In another example, the present disclosure provides another device. The device includes a probe shaft having a distal end and a proximal end. The probe shaft has a curved portion positioned between a distal portion of the probe shaft and a proximal portion of the probe shaft such that a longitudinal axis of a distal portion of the probe shaft has a non-zero angle with respect to a longitudinal axis of a proximal portion of the probe shaft. The proximal portion of the probe shaft includes a first tube having a first diameter and a second tube having a second diameter that is greater than the first diameter such that an air gap separates the first tube and the second tube. The device also includes a housing coupled to the proximal end of the probe shaft, and a handle coupled to the housing. The device also includes an end effector coupled to the distal end of the probe shaft. The end effector defines an atraumatic surface when the distal end of the probe shaft is advanced through a nasal cavity of a patient and is positioned proximate to a nasal tissue region having at least one nasal nerve. The end effector is configured to transmit lateral pressure against the nasal tissue region. The device also includes a trigger positioned in the handle. Activation of the trigger causes the end effector to ablate the at least one nasal nerve when the end effector is in contact against the nasal tissue region.

In yet another example, the present disclosure provides a method for treating a nasal tissue region of a nasal cavity of a patient. The method includes introducing a distal end of a probe shaft through the nasal cavity. The distal end of the probe shaft has an end effector with a first configuration having a low-profile which is shaped to manipulate tissue within the nasal cavity. The probe shaft has a curved portion such that a longitudinal axis of a distal portion of the probe shaft has a non-zero angle with respect to a longitudinal axis of a proximal portion of the probe shaft. A flexibility of the proximal portion of the probe shaft is greater than a flexibility of the distal portion of the probe shaft. The method also includes reconfiguring the end effector from the first configuration to a second configuration in which the end effector is shaped to contact and follow a contour of the nasal tissue region. The method also includes ablating, via the end effector, at least one nasal nerve of the nasal tissue region.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is an internal lateral view of the nasal cavity showing the relevant nasal anatomy and the associated nerves within and near the targeted region of the lateral nasal wall.

FIG. 2 is a perspective view of a device, according to an example.

FIG. 3 is top view of the device shown in FIG. 2, according to an example.

FIG. 4 is a top view of a distal end of the device shown in FIG. 2, according to an example.

FIG. 5 is a side view of an example cryogenic fluid source of the device shown in FIG. 2, according to an example.

FIG. 6 is a side view of the device shown in FIG. 2, according to an example.

FIG. 7 is a perspective cross-section view of the device shown in FIG. 2, according to an example.

FIG. 8 is a side cross-section view of a trigger of the device shown in FIG. 2, according to an example.

FIG. 9 is bottom view of the device shown in FIG. 2, according to an example.

FIG. 10A is a side view of an expandable member and planar member of an example end effector in a deflated configuration, according to an example.

FIG. 10B is a side view of an expandable member and planar member of an example end effector in an expanded configuration, according to an example.

FIG. 11 is a perspective view of the distal end of the probe shaft of the device shown in FIG. 2, according to an example.

FIG. 12A is a perspective view of the device shown in FIG. 2 including a temperature sensor, according to an example.

FIG. 12B is a perspective view of the device shown in FIG. 2 including a temperature sensor, according to another example.

FIG. 12C is a perspective view of the device shown in FIG. 2 including a temperature sensor, according to another example.

FIG. 12D is a perspective view of the device shown in FIG. 2 including a temperature sensor, according to another example.

FIG. 13 is a perspective view of the device shown in FIG. 2 including a camera and a light source, according to an example.

FIG. 14 is a perspective view of the device shown in FIG. 2 including a Doppler sensor, according to an example.

FIG. 15 is a perspective view of the device shown in FIG. 2 including an electrode, according to an example.

### DETAILED DESCRIPTION

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Illustrative, non-exhaustive examples, which may or may not be claimed, of the subject matter according the present disclosure are provided below.

The present disclosure is related to systems, devices, and methods for applying cryotherapy. More specifically, the present disclosure relates to applying cryotherapy for applications related to afflictions of the ear, nose, and throat. The devices and methods described herein can be particularly useful when delivering treatments to patients in an office-based setting. Use of the disclosed methods, devices, and systems can allow for improved delivery of cryotherapy treatments with more effectiveness and practicality relative to existing equipment and techniques.

Various aspects of the present disclosure described herein may be applied to any of the particular applications set forth below or for any other types of thermal or non-thermal treatment systems or methods. The present disclosure may be applied as a standalone system or method, or as part of an integrated medical treatment system.

Generally, the present disclosure seeks to improve at least some aspects of existing cryotherapy devices. The improvements described can enable better outcomes, more practical usage, and will ultimately benefit both patients and care providers.

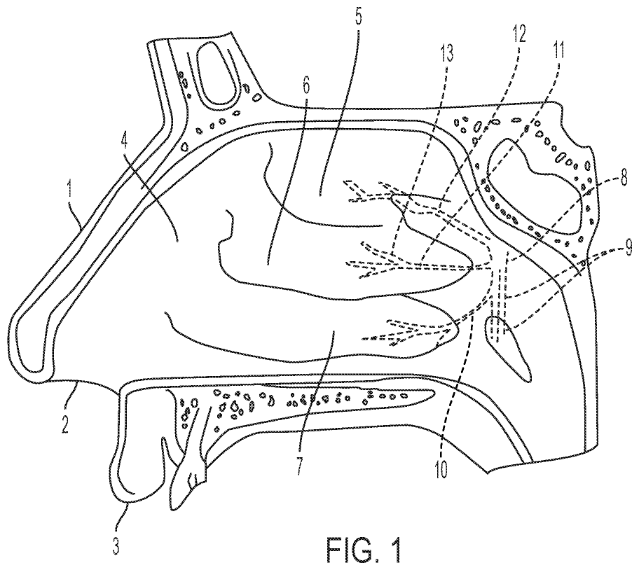

With reference to the Figures, FIG. 1 is an internal view of the nasal cavity showing some relevant nasal anatomy. Shown for orientation is a lateral nasal cavity wall 4, a nose 1, a nostril 2, and an upper lip 3. An superior turbinate 5, a middle turbinate 6, and an inferior turbinate 7 are depicted along with the associated nerves relevant to this disclosure shown in dashed lines. Posterior nasal nerves 10, 11 and 12 are responsible for the parasympathetic control of the nasal mucosa including the mucosa covering the turbinates. These posterior nasal nerves (PNNs) originate from the sphenopalatine ganglion. At times other accessory posterior nasal nerves (APNNs) may originate from the greater palatine canal or from the bony plate underneath the mucosa.

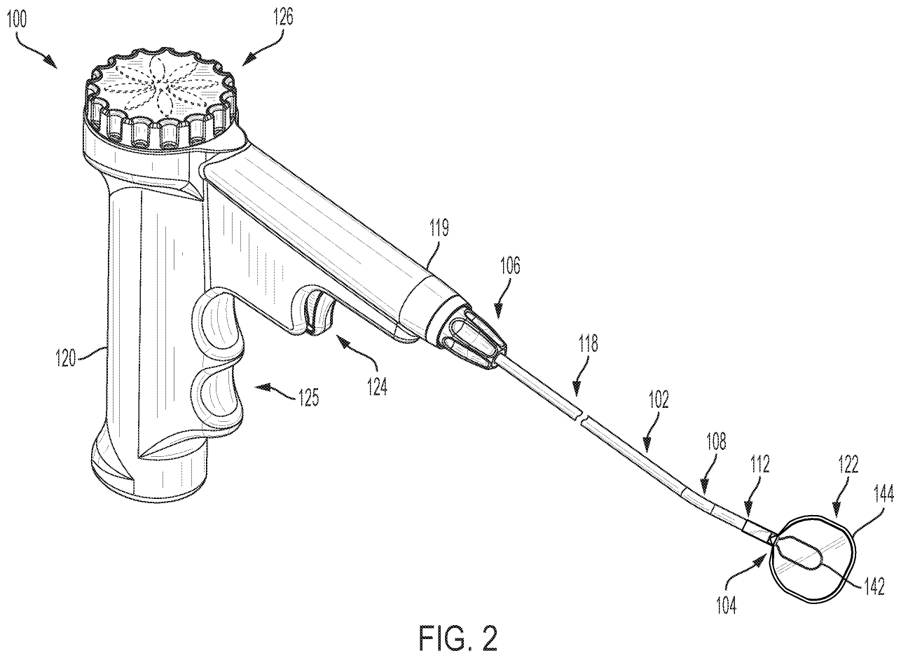

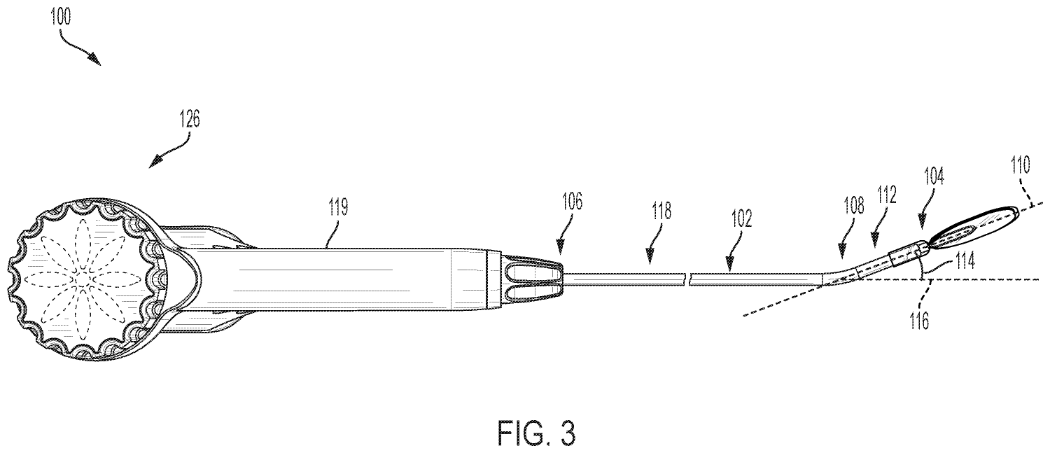

FIG. 2 is a schematic illustration of a device 100, which is configured for treatment of a nasal tissue region having at least one nasal nerve for the treatment of rhinitis and/or other conditions. As shown in FIG. 2, the device 100 includes a probe shaft 102 having a distal end 104 and a proximal end 106. As shown in the top view of the device 100 in FIG. 3, the probe shaft 102 has a curved portion 108 such that a longitudinal axis 110 of a distal portion 112 of the probe shaft 102 has a non-zero angle 114 with respect to a longitudinal axis 116 of a proximal portion 118 of the probe shaft 102. A flexibility of the proximal portion 118 of the probe shaft 102 can be greater than a flexibility of the distal portion 112 of the probe shaft 102, as discussed in additional detail below. As examples, a length of the proximal portion 118 of the probe shaft 102 is at least two times greater or at least three times greater than a length of the distal portion 112 of the probe shaft 102. The distal portion 112 of the probe shaft 102 can extend from the distal end 104 of the probe shaft 102 to the curved portion 108. The proximal portion 118 of the probe shaft 102 can extend from the proximal end 106 of the probe shaft 102 to the curved portion 108.

As shown in FIG. 2, the device 100 also includes a housing 119 coupled to the proximal end 106 of the probe shaft 102, and a handle 120 coupled to the housing 119. The proximal end 106 of the probe shaft 102 may extend into the housing 119. In one example, as shown in FIG. 2, the handle 120 includes a pistol grip including finger grips 125. As such, the device 100 may be configured to be held like a pistol by the practitioner using the handle 120 as shown in FIG. 2. Other arrangements for the handle 120 are possible as well.

The device 100 also includes an end effector 122 coupled to the distal end 104 of the probe shaft 102. In general, the end effector 122 is configured to ablate a target tissue adjacent to the end effector 122. For example, the end effector 122 can be configured to ablate at least one nasal nerve using cryogenic fluid (e.g., the end effector 122 can include a cryo-ablation element), radiofrequency (RF) energy, microwave energy, ultrasound energy, resistive heating, exothermic chemical reactions, or combinations thereof. Although the end effector 122 is described below for an implementation in which end effector 122 is configured to ablate the target tissue region using a cryogenic fluid, the end effector 122 can additionally or alternatively be configured to ablate the target tissue using one or more of the other ablation modalities described above. Additionally, the end effector 122 is shown having, multiple variations described herein and may be optionally interchanged depending upon which particular example utilized by a practitioner.

The end effector 122 can define an atraumatic surface when the distal end 104 of the probe shaft 102 is advanced through a nasal cavity of a patient and is positioned proximate to a nasal tissue region having at least one nasal nerve, for example the nasal nerve(s) associated with a lateral nasal wall. For example, the atraumatic surface of the end effector 122 can have a rounded and/or blunt edge, and omit pointed corners or sharp edges. To help define the atraumatic surface, the end effector 122 can additionally or alternatively be formed from a compliant material that can conform to a shape of anatomical structures contacted by the end effector 122 as the end effector 122 traverses through the nasal cavity. As examples, the end effector 122 can be formed, at least in part, from at least one material selected from among a group of materials including silicone rubber, a urethane rubber, nylon, and/or a polymeric material (e.g., polyethylene terephthalate (PET)).

Once positioned within the nasal tissue region, the end effector 122 is configured to transmit lateral pressure against the nasal tissue region. For example, the device 100 may be configured so that the practitioner can press the end effector 122 against the lateral nasal wall proximate to the target posterior nasal nerve. In some implementations, the end effector 122 can be configured to conform to the morphology of the target tissue (e.g., the lateral nasal wall) and to more evenly engage the target tissue (e.g., the lateral nasal wall) with a substantially uniform contact pressure as compared to an end effector 122 that does not conform to the morphology of the target tissue. This can help to effectively ablate the target tissue region in a relatively uniform manner and, thus, ablate the target tissue region in a more predictable and controllable manner to achieve a desired clinical outcome.

In one example, the probe shaft 102 may have a length between approximately 4 cm and approximately 10 cm, and a diameter between approximately 1 mm and approximately 4 mm. In some examples, the end effector 122 may have an outer diameter that approximates the diameter of the probe shaft 102. In other examples, the diameter of the end effector 122 may be larger or smaller than the diameter of the probe shaft 102. Additionally, in an example, the extended length of the end effector 122 may be between approximately 0.5 cm and approximately 1.5 cm. The end effector 122 can be substantially flexible along a longitudinal axis of the end effector 122 (e.g., along the axis 110); however, the end effector 122 may also be at least partly malleable and configured for form shaping, by the user. Form shaping of the end effector 122 may be performed manually by the practitioner. Various lengths, shapes, and diameters of the end effector 122 of the device 100 may be produced and supplied to the end user.

Within examples, the end effector 122 can be additionally or alternatively configured to transmit the lateral pressure against the nasal tissue region based on at least one feature selected from among a group of features including: (i) the probe shaft 102 having the curved portion 108 such that the longitudinal axis 110 of the distal portion 112 of the probe shaft 102 has a non-zero angle with respect to the longitudinal axis 116 of the proximal portion 118 of the probe shaft 102, and (ii) the flexibility of the proximal portion 118 of the probe shaft 102 being greater than a flexibility of the distal portion 112 of the probe shaft 102.

For instance, due to the curved portion 108, the proximal portion 118 of the probe shaft 102 can allow the end effector 122 to contact and applanate against the nasal tissue region of interest while the proximal portion 118 of the probe shaft 102 applies negligible or no pressure against other anatomical features of the nasal cavity. As shown in FIG. 3, the non-zero angle 114 between the longitudinal axis 110 of the distal portion 112 of the probe shaft 102 and the longitudinal axis 116 of the proximal portion 118 of the probe shaft 102 can be between about 15 degrees and about 25 degrees, and preferably about 20 degrees. Such a bend in the probe shaft 102 at the curved portion 108 can additionally or alternatively facilitate navigation of the end effector 122 through the nasal cavity and allows for improved maneuverability around and against structures such as the middle and inferior turbinates.

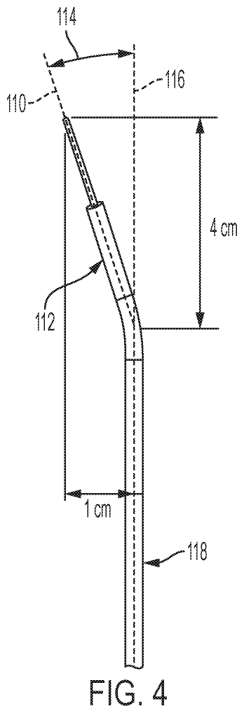

In one implementation of the device 100, as shown in FIG. 4, the curved portion 108 of the probe shaft 102 is positioned about 4 cm from the distal end of end effector 122 of the probe shaft 102, and the curved portion 108 of the probe shaft 102 causes a lateral deviation of the distal end of end effector 122 of the probe shaft 102 with respect to the longitudinal axis 116 of the proximal portion 118 of the probe shaft 102 of about 1 cm. It has been found that positioning the curved portion 108 of the probe shaft 102 about 4 cm form the distal end of the end effector 122 can beneficially help to target the inferior turbinate using the device 100. For procedures targeting a different tissue region, the curved portion 108 can be positioned at a different distance relative to the distal end of the end effector 122. With the presently-disclosed example, an improved (or optimized) navigation capability has been created, and there is an improved ability to make sufficient contact between the end effector 122 and key anatomical structures within the nasal cavity.

Additionally, as noted above, the flexibility of the proximal portion 118 of the probe shaft 102 can be greater than a flexibility of the distal portion 112 of the probe shaft 102. This difference in flexibility between the proximal portion 118 of the probe shaft 102 and the distal portion 112 of the probe shaft 102 can provide a flexing location of the probe shaft 102 at a location between the proximal portion 118 and the distal portion 112 (e.g., at the curved portion 108 of the probe shaft 102) when the end effector 122 engages the target tissue region. The flexing location between the proximal portion 118 and the distal portion 112 can be more proximally located along the probe shaft 102 than a flexing location of the probe shaft 102 in implementations in which the probe shaft 102 does not have a difference in flexibility between the proximal portion 118 and the distal portion 112. Providing the flexing location more proximally along the probe shaft 102 can allow for a relatively large portion (e.g., greater than 50 percent) or an entirety of a tissue-facing surface of the end effector 122 to more evenly contact a surface of a target tissue (e.g., the lateral nasal wall) when a practitioner manipulates the handle 120 in a direction towards the target tissue, as compared to implementations in which the probe shaft 102 has substantially the same flexibility over an entire length of the probe shaft 102.

Within examples, to provide the difference in flexibility between the proximal portion 118 and the distal portion 112 of the probe shaft 102, the proximal portion 118 and the distal portion 112 of the probe shaft 102 can (i) be formed from different material(s) and/or (ii) have different dimensions. For instance, the proximal portion 118 can be formed from one or more rigid materials selected from among: metal tubing (ie stainless steel tubing), polymeric/plastic tubing (ie PEEK, Nylon, ABS, Urethane, polyethylene), and woven/braided tubing. The distal portion 112 each can be formed from one or more materials selected from among: a thermoplastic elastomer (e.g., polyether block amide also known as PEBAX), nylon, urethane, polyethylene, polyether ether ketone (PEEK), polytetrafluoroethylene (PTFE), laser cut metal tubing, metal coiling material, and mesh/braided shaft material. Additionally, for instance, the one or more materials selected for the proximal portion 118 can be different than the one or more materials selected for the distal portion 112.

In one example, the distal portion 112 of the probe shaft 102 can have a flexibility that is approximately two times to approximately four times greater than a flexibility of the proximal portion 118 of the probe shaft 102. In an implementation, the distal portion 112 can have a respective hardness value selected from a range of values between approximately 35 Shore D and approximately 72 Shore D.

Additionally, in an example, the distal portion 112 of the probe shaft 102 can have respective stiffness and/or flexibility values such that a force required to bend the distal portion 112 and the end effector 122 by approximately 22 degrees relative to the proximal portion 118 of the probe shaft can be between 0.3 pounds and approximately 0.7 pounds. In another example, the distal portion 112 of the probe shaft 102 of the probe shaft 102 can have respective stiffness and/or flexibility values such that a force required to bend the distal portion 112 and the end effector 122 by approximately 22 degrees relative to the proximal portion 118 of the probe shaft can be between 0.6 pounds and approximately 0.7 pounds. In another example, the distal portion 112 of the probe shaft 102 can have respective stiffness and/or flexibility values such that a force required to bend the distal portion 112 and the end effector 122 by approximately 22 degrees relative to the proximal portion 118 of the probe shaft can be between 0.3 pounds and approximately 0.5 pounds.

The probe shaft 102 may be configured to be rotatably coupled to the housing 119 of the device 100 to facilitate positioning of the end effector 122 without having to rotate the device 100 excessively. In one example, the probe shaft 102 is rotatable 180 degrees with respect to the housing 119 of the device 100. As such, the non-zero angle 114 between the longitudinal axis 110 of the distal portion 112 of the probe shaft 102 and the longitudinal axis 116 of the proximal portion 118 of the probe shaft 102 may be adjustable from angling to the left when looking at the device 100 from a top view, to angling to the right when looking at the device 100 from a top view. For example, during use the practitioner may insert the end effector 122 of the device 100 and ablate a target nasal nerve in the left nostril of the patient, remove the device from the patient's nasal cavity, rotate the probe shaft 102 180 degrees, and then insert the end effector 122 of the device 100 and ablate a target nasal nerve in the right nostril of the patient without modifying the practitioner's grip on the handle 120.

In one particular example, the housing 119 of the device 100 just proximal to the proximal end 106 of the probe shaft 102 may include a pair of detents and a corresponding pair of cutouts. The pair of detents may be positioned approximately 180 degrees apart, and the corresponding pair of cutouts may also be positioned approximately 180 degrees apart. In a first configuration (e.g., a configuration in which the probe shaft 102 angles to the left when looking at the device 100 from a top view), a first detent of the pair of detents is positioned in a first cutout of the pair of cutouts, and a second detent of the pair of detents is positioned in a second cutout of the pair of cutouts. Upon rotation of the probe shaft 102, the pair of detents may be configured to rotate with respect to the pair of cutouts until the device 100 is in a second configuration. In the second configuration, (e.g., a configuration in which the probe shaft 102 angles to the right when looking at the device 100 from a top view), the first detent is positioned in the second cutout, and the second detent is positioned in the first cutout.

The device 100 also includes a trigger 124 positioned in the handle 120. Activation of the trigger 124 causes the end effector 122 to ablate the at least one nasal nerve in the nasal tissue when the end effector 122 is in contact against the nasal tissue region. The at least one nasal nerve of the nasal tissue region can include one or more of a posterior nasal nerve of a nasal branch of a vidian nerve, as a non-limiting example. In another example, the distal end 104 of the probe shaft 102 is advanced through the nasal cavity of the patient and in proximity of a sphenopalatine foramen. As noted above, the difference in flexibility between the proximal portion 118 of the probe shaft 102 and the distal portion 112 of the probe shaft 102 causes a flexing location of the probe shaft 102 to shift to a more proximal location on the device 100, allowing the end effector 122 to lay against a flat surface such as the lateral nasal wall as described above. This difference in flexibility additionally or alternatively enables the device 100 to accommodate a larger range of anatomies without requiring the operator to apply inappropriately large tissue forces in order to establish proper tissue contact.

As noted above, the end effector 122 can be configured to ablate the at least one nasal nerve using at least one ablation modality selected from among a group of modalities including: cryogenic fluid (e.g., a cryo-ablation element), RF energy, microwave energy, ultrasound energy, resistive heating, exothermic chemical reactions, or combinations thereof. In one example, the device 100 includes a cryogenic fluid source 126 positioned at least partially in the handle 120, and a lumen disposed in the probe shaft 102 and in fluid communication with the cryogenic fluid source 126. In one example, the cryogenic fluid source 126 may be supplied with liquid cryogen and configured for a single patient use.

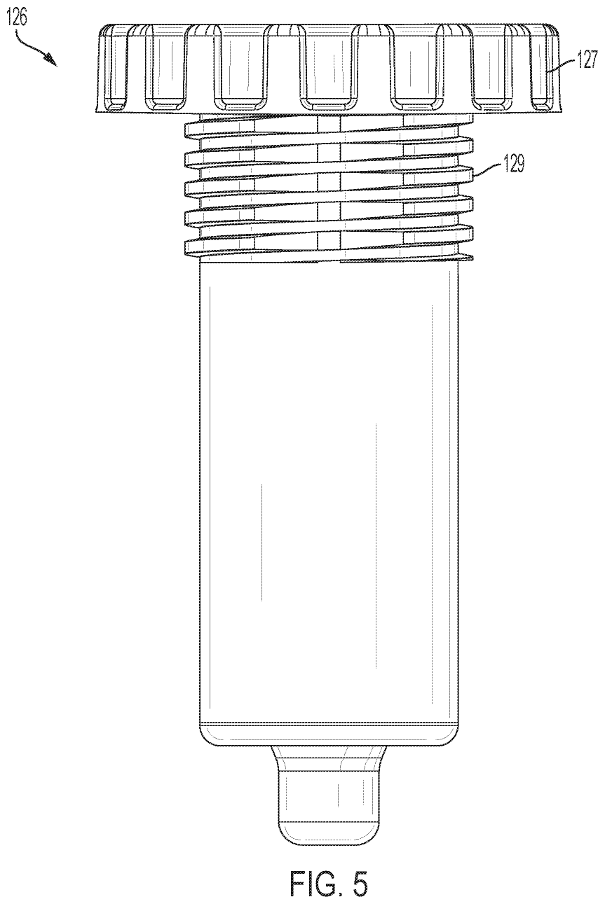

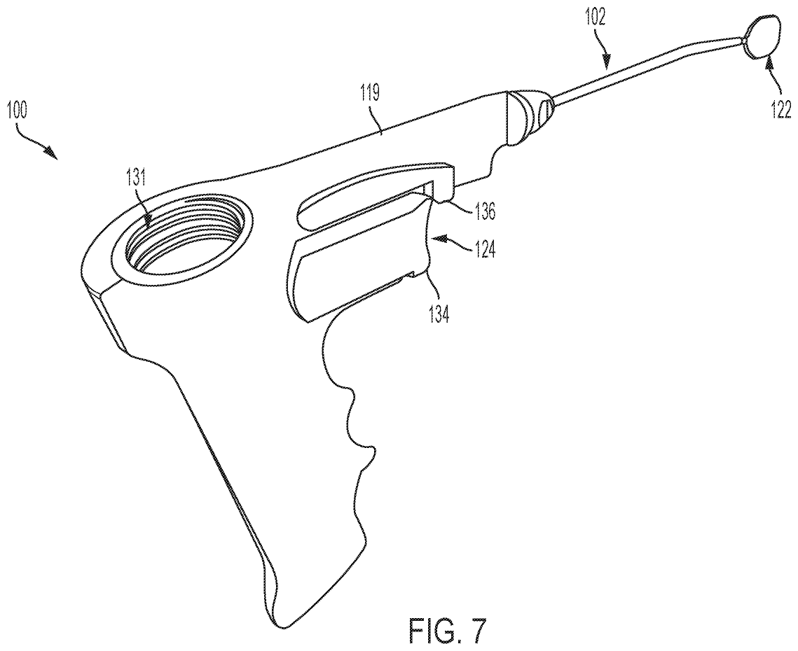

Alternatively, the device 100 may be configured for use with a user replaceable cryogenic fluid source 126 in the form of a canister that is removably positioned at least partially in the handle 120. Such an example canister is illustrated in FIG. 5. As shown in FIG. 5, the cryogenic fluid source 126 includes a cap 127 and a plurality of threads 129 configured to interact with a plurality of threads 131 of the handle 120 (see FIG. 7) to thereby removably couple the cryogenic fluid source 126 to the device 100. In yet another alternative, a reservoir separate from the device 100 may be fluidly coupled to the handle 120. In such an example, the device 100 further includes a liquid cryogen flow control valve, not shown, that may be disposed in fluidic communication with the cryogenic fluid source 126 and the lumen in the probe shaft 102.

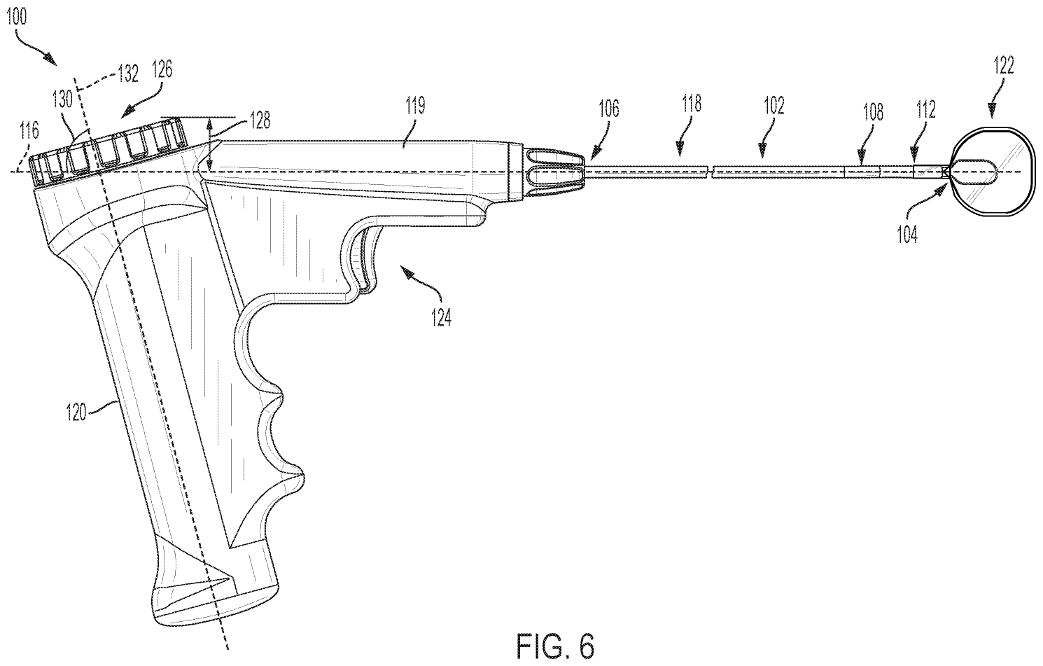

FIG. 6 is a side view of the device, which illustrates a height 128 of the cryogenic fluid source 126 relative to the longitudinal axis 116 of the proximal portion 118 of the probe shaft 102. In one example, the height 128 is less than approximately 2 cm. In another example, the height 128 can be approximately 0.5 inches (e.g., approximately 1.27 cm). A height of this size enables all the necessary device elements, including the cryogenic fluid source 126 and associated cryo-line input features, to fit in the device 100 in an orientation that enables adequate outflow, while at the same time allowing for enough grip space for a user to rotate a cap of the cryogenic fluid source 126 with sufficient torque for placement/puncture of the cryogen canister and for subsequent removal of the canister following treatment. Reducing the height 128 provides several advantages to the convenience of the operator and ultimately to the likelihood of procedural success, as a reduced height allows for the operator to hold the device in one hand and have a second hand operate an endoscope (or other tool) simultaneously with little to no interference. More specifically, the reduced height 128 allows for the secondary hand operating an endoscope or other tool to freely cross the plane of the device hand when navigating the device 100 into the nasal cavity.

In addition, as shown in FIG. 6, the device 100 includes an angle 130 between a longitudinal axis 132 of the cryogenic fluid source 126 and the longitudinal axis 116 of the proximal portion 118 of the probe shaft 102. In an example, the angle 130 between the longitudinal axis 132 of the cryogenic fluid source 126 and the longitudinal axis 116 of the proximal portion 118 of the probe shaft 102 can be configured to allow for a flow of the cryogenic fluid from the cryogenic fluid source 126 to the end effector 122 both while the patient is sitting upright and while the patient is laying prone. In example implementations, the angle 130 longitudinal axis 132 of the cryogenic fluid source 126 and the longitudinal axis 116 of the proximal portion 118 of the probe shaft 102 may range between about 0 degrees to about 90 degrees, between about 10 degrees and about 90 degrees, between about 20 degrees and about 90 degrees, between about 30 degrees and about 90 degrees, between about 40 degrees and about 90 degrees, between about 50 degrees and about 90 degrees, between about 60 degrees and about 90 degrees, between about 60 degrees and about 100 degrees, and between about 70 degrees and about 90 degrees. In another implementation, the angle 130 can be about 75 degrees to facilitate treating patients who are lying completely flat as well as patients who are sitting completely upright. Further, an approximately 75 degree relative angle between the longitudinal axis 132 of the cryogenic fluid source 126 and the longitudinal axis 116 of the proximal portion 118 of the probe shaft 102 also accounts for the position of the patient's head in relation to the patient's body. As such, the presently-disclosed design allows for improved (or optimal) flexibility and freedom for a provider to treat patients in the largest number of positions.

With reference to FIG. 7, examples of the presently-disclosed device 100 include a trigger 124 enables a simplified operation that a user can accomplish reliably using a single hand or single finger. As shown, implementations include a trigger-type toggle valve 134 that can be squeezed by a user to initiate cryogen release through the probe shaft 102 into the end effector 122.

Additionally, in FIG. 7, the trigger 124 includes a lockout lever 136. In an implementation, the lockout lever 136 can be biased towards the toggle valve 134 (e.g., by a torsion spring). Response to depressing the toggle valve 134 from an initial position towards the handle 120, the lockout lever 136 can clear and extend distal to the toggle valve 134, thereby preventing the toggle valve 134 from releasing back to the initial position. While the lockout lever 136 impedes the toggle valve 134, the cryogenic fluid can continue to flow from the cryogenic fluid source 126 to the end effector 122. To terminate the release of cryogen, a user may move a lockout lever 136 against the biasing force so that the toggle valve 134 can return to the initial position.

In some implementations, the practitioner may apply approximately four pounds of force to depress the toggle valve 134 and cause the cryogenic fluid to flow to the end effector 122. During some procedures, the practitioner may maintain this force on the toggle valve 134 for approximately 30 seconds for each nostril of a given patient, and may perform this procedure on multiple patients in a given day. Accordingly, the lockout lever 136 can help to mitigate fatigue on the fingers of the practitioner operating the device 100 by allowing the cryogenic fluid to continue to flow without the practitioner maintaining the force on the toggle valve 134 for an entirety of the procedure. Although the lockout lever 136 can provide such benefits, the device 100 can omit the lockout lever 135 in some alternative implementations.

In examples, the toggle valve 134 and lockout lever 136 are located proximate to handle 120 in a position such that all adult operators are expected to be able to reach the toggle valve 134 with a finger on the same hand which grips the handle 120. As a result of these improvements over existing devices, the presently-disclosed device 100 can now be suitably operated with a single hand. As such, the device 100 may be configured so that it is held by the user like a pistol having a pistol grip where the toggle valve 134 is configured like a pistol trigger. Other example arrangements are possible as well.

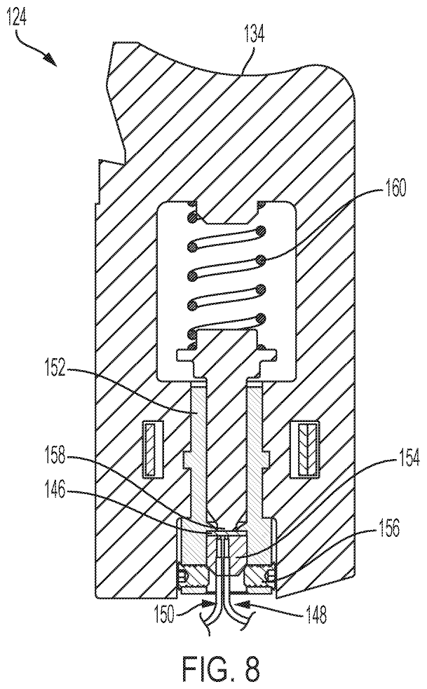

FIG. 8 illustrates a cross-sectional view of an example trigger 124 of the device 100 using positive pressure from a nitrous oxide canister to lift a membrane 146 allowing for flow between a proximal cryo-line 148 and a distal cryo-line 150. As shown in FIG. 8, the trigger 124 includes a valve housing 152, a valve plug 154, a membrane 146, set screws 156, a valve stem 158, a toggle valve 134, and a trigger spring 160. The set screws 156 in the valve housing 152 force the valve plug 154 and the membrane 146 to be in intimate contact with each other creating a seal around the perimeter of the valve housing 152. In its default state, the trigger 124 is in the closed position with the trigger spring 160 and valve stem 158 providing sufficient force to seal the membrane 146 against the face of the valve plug 154 where the hole to the proximal cryo-line 148 is located. When the toggle valve 134 is pressed, the valve housing 152, valve plug 154, and membrane 146 move away from the valve stem 158. Once the trigger 124 has moved a sufficient distance from the valve stem 158, the force from the pressurized nitrous oxide becomes sufficient to break the seal of the membrane 146 with the hole to the proximal cryo-line 148 located in the valve plug 154. This allows for the membrane 146 to dome, creating a pressurized space that connects the proximal cryo-line 148 and the distal cryo-line 150. Releasing the toggle valve 134 forces the valve housing 152, valve plug 154, and membrane 146 to return to make contact with valve stem 158 at a rate defined by the trigger spring 160, to close on the membrane 146 and valve plug 154 proximal cryo-line 148.

As shown in FIG. 8, the distal cryo-line 150 may have an inner diameter that is smaller than the inner diameter of the proximal cryo-line 148. Such an arrangement ensures that the space under the membrane 146, when it is in the open position, experiences improved pressurization due to the extra resistance from the smaller inner diameter distal cryo-line 150. Improved pressurization by the distal cryo-line 150 reduces the pressure drop proximal to said distal cryo-line 150 and allows for the liquid cryogen to be utilized more efficiently.

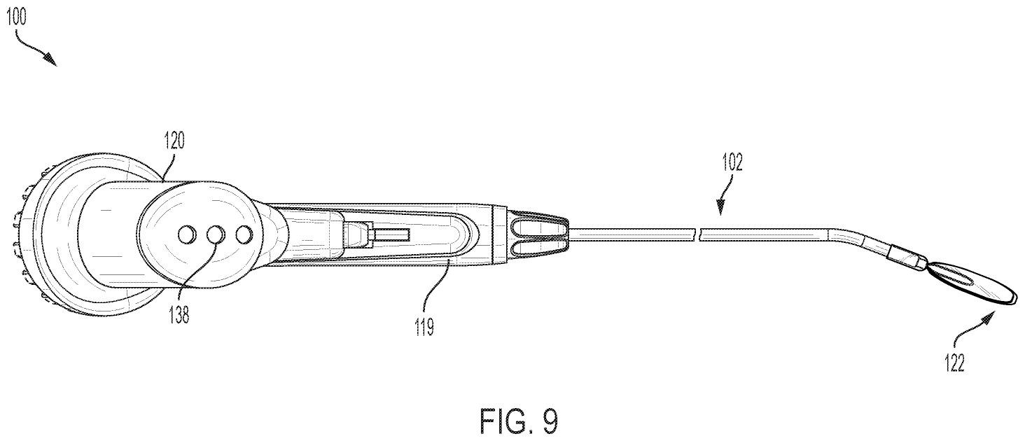

The pressurized cryogenic fluid source 126 may contain a liquid cryogen, e.g., nitrous oxide, but may also be another cryogenic liquid such as liquid carbon dioxide, or a liquid chlorofluorocarbon compound, etc. In use, liquid cryogen is introduced into the end effector 122 through a liquid cryogen supply line that is connected to the cryogenic fluid source 126 in the handle 120, and runs coaxially through the probe shaft 102. The end effector 122 is configured as a liquid cryogen evaporator, and is configured to be pressed against the lateral nasal wall proximate to the SPF as described above for cryo-ablation of at least one posterior nasal nerve. The construction and the function of the end effector 122, and alternative examples are described in detail below. The evaporated liquid cryogen may be vented to the room, e.g., through the probe shaft 102 to one or more vent ports 138 in the handle 120 (shown in FIG. 9), or in the vicinity of the proximal end 106 of the probe shaft 102. As such, no liquid or gas cryogen is introduced into the patient's nasal cavity.

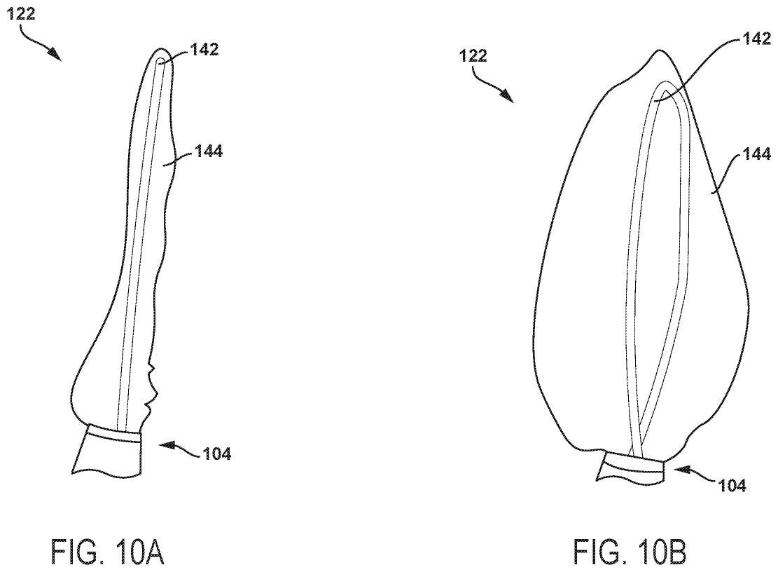

In one example of the present disclosure, as shown in FIGS. 10A-10B, the end effector 122 of the device 100 includes a planar member 142 defining a flattened shape disposed at the distal end 104 of the probe shaft 102, and an expandable structure 144 surrounding the planar member 142 and coupled to the distal end 104 of the probe shaft 102. The planar member 142 includes an elongate structure with arcuate edges to define an atraumatic surface. The expandable structure 144 is inflatable from a deflated configuration (shown in FIG. 10A) to an expanded configuration (shown in FIG. 10B). An interior of the expandable structure 144 is in fluid communication with the cryogenic fluid source 126. The expandable structure 144 is configured to transition from the deflated configuration to the expanded configuration upon evaporation of cryogenic fluid within the interior of the expandable structure 144. In use, the end effector 122 formed by the planar member 142 and expandable structure 144 is configured as cryogenic evaporation chamber, and the outer surface of expandable structure 144 is configured as a cryo-ablation surface. The expandable structure 144 is configured apply a force against the lateral nasal wall between approximately, e.g. 20 grams and 200 grams.

The expandable structure 144 may be formed from an elastomeric material such as silicone rubber, or a urethane rubber. Alternatively, the expandable structure 144 may be formed from a substantially non-elastomeric material such as nylon or PET. In an example, the expandable structure 144 is configured to expand to a predetermined shape and size in the expanded configuration, and the predetermined shape and size corresponds to a shape and size of the nasal tissue region to be targeted for treatment. For instance, the expandable structure 144 is configured so the shape and the size of the structure matches the shape and the size of the cul-de-sac of the middle meatus defined by the tail of the middle turbinate, the middle turbinate, the lateral nasal wall, and the inferior turbinate, which is an example target location for the ablation of the posterior nasal nerves for the treatment of rhinitis. Matching the size and shape of the expandable structure 144 to the size and shape of the target anatomy facilitates improved tissue freezing and ablation of posterior nasal nerves. The expandable structure 144 may have an expanded diameter between approximately 3 mm and 12 mm in one radial axis, and may be configured such that the expanded diameter in one radial axis is different than another radial axis. The planar member 142 may include an elongate loop structure formed by a rigid wire that is configured to manipulate tissue in the nasal cavity. Further, the planar member 142 may be coupled to the distal end 104 of the probe shaft 102 within such that the planar member 142 is unattached to an interior of the expandable structure 144. In use, the device 100 is configured to cool an external surface of the expandable structure 144 to between −20 degrees Celsius (C) to −90 degrees C. for less than 120 seconds so as to controllably freeze the at least one nasal nerve at a depth less than 4 mm from a surface of the lateral nasal wall tissue region so as to reduce at least one symptom of rhinitis of the patient.

In some examples of the present device 100, the planar member 142 can assume a wide shape that tracks the perimeter of the expandable structure 144. Also, in some examples, the planar member 142 can couple to the probe shaft 102 approximately 15 mm proximal to the expandable structure 144. As illustrated in FIGS. 10A-10B, with the aforementioned changes to the shape of the planar member 142 and the attachment configuration of the expandable structure 144, the magnitude of expansion of the expandable structure 144 may be improved and may result in a greater degree of bilateral expansion (i.e., the expandable structure 144 extends away from the planar member 142 in both directions). Further, the geometry of the planar member 142 and the expandable structure 144 may enhance tissue contact, particularly in treatment regions such as the middle meatus, where it may be desirable to simultaneously treat the lateral nasal wall as well as portions of the middle turbinate itself.

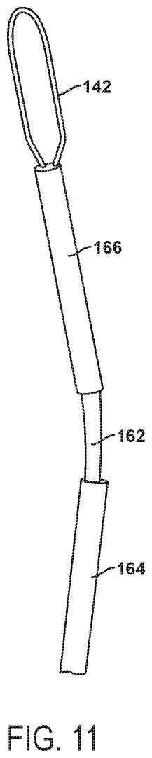

FIG. 11 illustrates an improved insulation system for the probe shaft 102, according to one example. In particular, in addition to a polymer insulation layer coating the exterior of the cannula (not shown in FIG. 11), a two-tube system may be used. As shown in FIG. 11, the proximal portion 118 of the probe shaft 102 includes a first tube 162 having a first diameter, and a second tube 164 having a second diameter that is greater than the first diameter such that an air gap separates the first tube 162 and the second tube 164. During cryotherapy, the cryogen exhaust travels through the smaller, inner first tube 162. This smaller first tube 162 is covered by a larger second tube 164 such that an air gap separates the two tubes. As mentioned above, a polymer insulation layer covers the entire complex. The result is increased insulation of the exterior surface of the probe shaft 102 from the inner exhaust tube (e.g., the first tube 162), and as a consequence little to no temperature changes are noted at the exterior of the probe shaft 102 during use.

Preferred implementations of such an insulated system may utilize hypotubes comprised of stainless steel or other similar materials. Stainless steel provides sufficient mechanical strength while simultaneously allowing for a minimal thickness of the tube wall. Limiting the thickness of the tube wall enables the size of the air gaps between adjacent tubes to be maximized, thus maximizing insulation. In one example, the inner first tube 162 may have an inner diameter of approximately 0.046 inches with an outer diameter of approximately 0.056 inches. An inner diameter of this size ensures sufficient area for cryogen exhaust to flow through the internal tube lumen in order to achieve a desired pressure within the end effector 122. An outer diameter of this size may help prevent kinking of the first tube 162 during use. In one example, the outer second tube 164 has an inner diameter of approximately 0.085 inches with an outer diameter of approximately 0.095 inches. The outer second tube 164 outer diameter of the size described minimizes the profile of the probe shaft 102 for navigation within the nasal cavity, with the inner diameter of this outer second tube 164 again selected in order to prevent kinking of the tube. In the example described, the resulting air pocket for insulation is approximately 0.014-0.015 inches. In preferred implementations, the first tube 162 and the second tube 164 are centered at the distal and proximal edges. A material such as stainless steel provides the additional benefit of ensuring that the first tube 162 and the second tube 164 maintain their relative spacing separation, thus maximizing insulation and preventing cold spots.

The probe shaft 102 may be fabricated from various biocompatible materials. In one example, the distal portion 112 of the probe shaft 102 comprises a first material, and the proximal portion 118 of the probe shaft 102 comprises a second material that is different than the first material. In one example, the first material comprises a polymer, and the second material comprises stainless steel. Such a difference in material may provide the difference in flexibility between the proximal portion 118 of the probe shaft 102 and the distal portion 112 of the probe shaft 102, as discussed in additional detail below. FIG. 11 illustrates the distal end 104 of the probe shaft 102 in such an example.

In particular, FIG. 11 illustrates the distal end 104 of the probe shaft 102 as a multi-lumen polymer tube 166 that resides between the proximal portion 118 of the probe shaft 102 (shown as the inner first tube 162) and the planar member 142. Further from the distal end 104 of the probe shaft 102, the inner first tube 162 enters into a larger outer second tube 164 which surrounds the inner first tube 162, as discussed above. The first tube 162 and the second tube 164 may comprise stainless steel, as a non-limiting example. The paddle legs of the planar member 142 may be laser welded into place after traveling through the flexible polymer tube 166. This configuration maintains the desired rigidity in the plane of the planar member 142 and continues to provide a sealed inner lumen for exhaust, but increases flexibility in the plane of anticipated tissue contact due to the inherent flexibility of the polymer tube 166. In other words, bending of the end effector 122 can begin more proximal along the probe shaft 102, allowing for a similar degree of bend to be achieved with less overall force applied.

In examples of the presently-disclosed device 100, the planar member 142 may be constructed of stainless steel wire having a diameter range of about 0.010 to about 0.020 inches, with a preferred diameter of 0.015 inches. In examples, the wire is shaped so as to ensure the wire doesn't obstruct the cryogen spray emerging from the probe shaft 102 and so that the wire is narrowed proximal of the planar member 142 so as to minimize the profile of the structure. The shape of the planar member 142 shown in FIG. 2 is one example of a suitable shape, but it will be apparent to those skilled in the art that alternative shapes are possible without loss of novelty. In some examples, the legs of the planar member 142 may range between about 5 to about 50 mm in length, with a preferred length of approximately 30 mm.

In examples of the presently-disclosed device 100, the wire legs of the planar member 142 may be inserted into a tube, for example a three-lumen polymer tube 166. Each leg may insert into an independent lumen that is sized appropriately to provide a tight fit around the wire. In examples, the central lumen may remain open to be employed for other device purposes, such as an exhaust lumen for evaporated cryogen material. In variation examples, the polymer tube 166 may contain fewer than three or greater than three lumens. In some examples, the polymer tube 166 is placed such that its distal end touches the proximal end of the planar member 142. The polymer tube 166 is preferably constructed of a thermoplastic elastomer having a hardness in the range of 40-80 shore D or another suitable polymer material that retains appropriate flexibility while maintaining an ability to be thermally-processed and attached to similar materials. In preferable examples, the polymer tube 166 has a length of approximately 20 mm. In one example, during device construction, the proximal end of the central lumen of the polymer tube 166 is pressed onto a curved rigid proximal portion 118 of the probe shaft 102 so that the polymer tube 166 overlaps the proximal portion 118 of the probe shaft 102 between about 2 mm to about 7 mm. The wire legs of the planar member 142 may then be affixed to the probe shaft 102 via laser welding or a similar technique. In examples, an inner first tube 162 runs the entire length of the probe shaft 102 and is affixed to a larger outer second tube 164 inside the handle 120. As discussed above, this construction allows for a 10-15 mm flexible and incompressible device neck that retains a sealed inner lumen for cryogen exhaust.

The presence of the polymer tube 166 at the distal end 104 of the probe shaft 102 results in an unexpectedly large reduction in force needed to position the planar member 142 flush against a flat surface. In particular, presently-disclosed device may require less than 4 ounces of force to position the planar member 142 flat on a surface, and preferably less than about 2 ounces of force. With the incorporation of the novel design aspects disclosed herein, the flexing location of the probe shaft 102 shifts to a more proximal location on the device 100, allowing the entire planar member 142 to lay against a flat surface such as the lateral nasal wall. This enables the device 100 to accommodate a larger range of anatomies without requiring the operator to apply inappropriately large tissue forces in order to establish proper tissue contact.

Additional examples of exemplary devices are described below. The features of any of the devices or device components described in any of the examples herein can be used in any other suitable example of a device or device component. In one example, the present disclosure provides a surgical probe which is configured for ablation where the surgical probe includes a surgical probe shaft comprising an elongated structure with a distal end and a proximal end, an expandable structure attached to the distal end of the probe shaft, the expandable structure having a deflated configuration and an expanded configuration, a member attached to the distal end and extending within the expandable structure such that the member is unattached to an interior of the expandable structure, wherein the member defines a flattened shape which is sized for placement against a lateral nasal wall proximate to a posterior nasal nerve, and a lumen in fluid communication with the interior of the expandable structure.

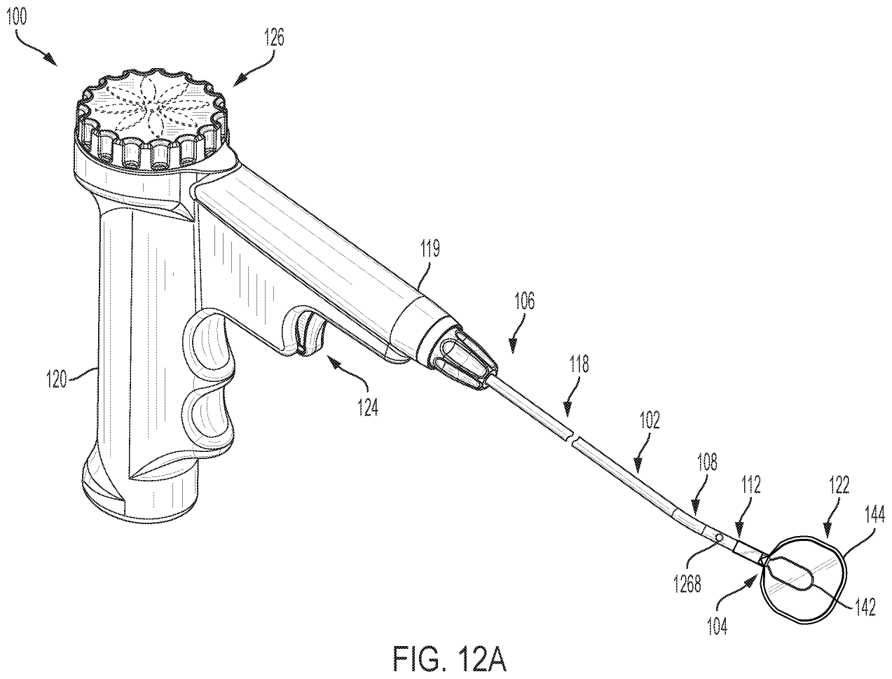

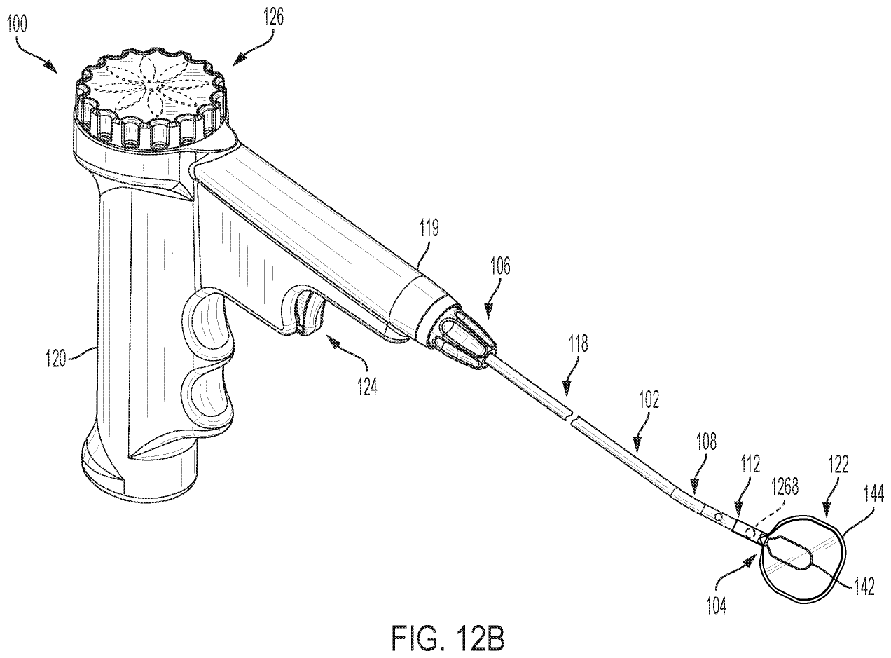

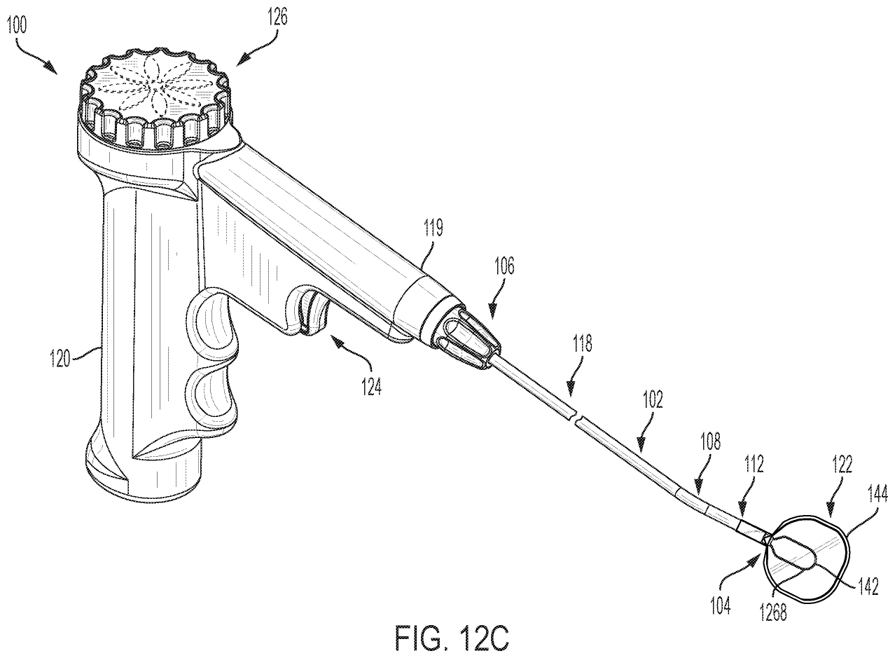

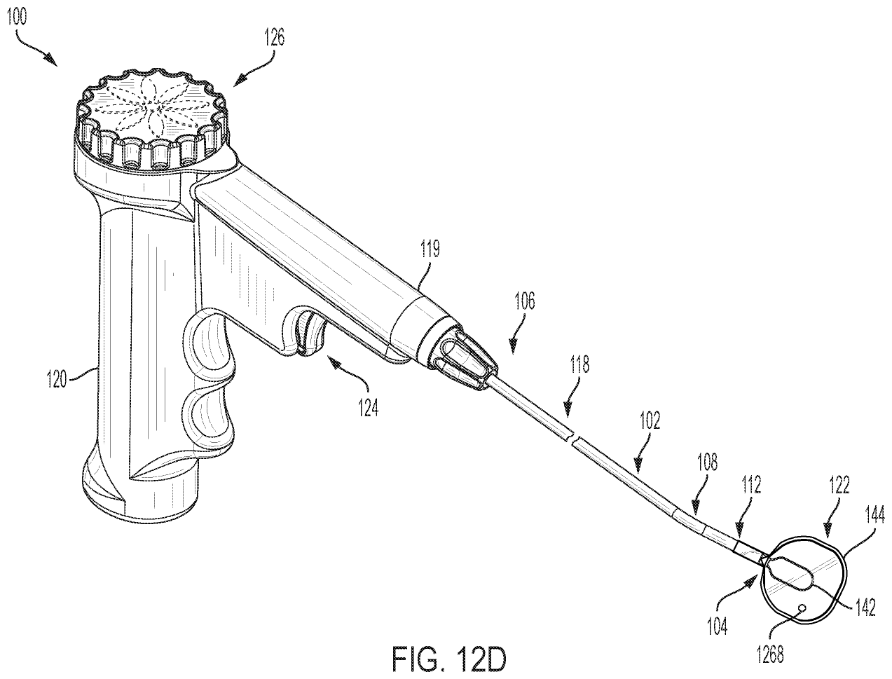

The device 100 may be configured as a simple mechanical device that is void of electronics as shown. Alternatively, device 100 may be configured with at least one electronic function. In one example, a temperature sensor may be disposed in the vicinity of the end effector 122. As examples, FIG. 12A-12D depicts the device 100 shown in FIGS. 2-11 including a temperature sensor 1268 in various locations. In general, the temperature sensor 1268 can measure a temperature and generate a signal indicative of the temperature. Within examples, the device 100 can be configured to take one or more actions based on a temperature sensed by the temperature sensor 1268.

In FIG. 12A, the temperature sensor 1268 is located on an exterior of the probe shaft 102 at a location that is proximal to the end effector 112. In an example, the temperature sensor 1268 locate on the exterior of the probe shaft 102 and proximal to the end effector 112 can help to determine if a cryogenic cooling treatment has expanded outside of a desired target area. For instance, if the temperature sensor 1268 senses a temperature below a threshold temperature, it may be indicative that the device 100 should cease supplying the cryogen to the end effector 122. In some implementations, the temperature sensor 1268 and/or a controller can be configured to automatically cease a supply of the cryogen to the end effector 122 responsive to the temperature sensor 1268 sensing that the temperature is below the threshold temperature.

In FIG. 12B, the temperature sensor 1268 is located in an interior of the probe shaft 102 at a location that is proximal to the end effector 112. In an example, the temperature sensor 1268 located in the interior of the probe shaft 102 and proximal to the end effector 112 can sense a temperature that can be indicative of whether the cryogen is being fully converted from a liquid phase to a gas phase. For instance, the temperature sensor 1268 and/or a controller can determine that the cryogen is not being fully converted from a liquid to a gas, and the cryogen is flowing from the end-effector 122 to the handle 120 as a liquid responsive to the temperature sensor 1268 determining that the temperature sensed by the temperature sensor 1268 is less than a threshold temperature. As an example, the threshold temperature can be approximately negative 88 degrees Celsius.

In FIG. 12C, the temperature sensor 1268 is located in an interior space of the expandable structure 144 of the end effector 122. More particularly, in FIG. 12C, the planar member 142 is a thermocouple that provides both the structural functions and the temperature sensing functions described above. Similar to the temperature sensor 1268 located in the interior of the probe shaft 102, the temperature sensor 1268 located in the interior space of the expandable structure 144 of the end effector 122 can help to determine whether cryogen is being fully converted from a liquid to a gas. For instance, the temperature sensor 1268 and/or a controller can determine that the cryogen is not being fully converted from a liquid to a gas, and the cryogen is flowing from the end-effector 122 to the handle 120 as a liquid responsive to the temperature sensor 1268 determining that the temperature sensed by the temperature sensor 1268 is less than a threshold temperature. As an example, the threshold temperature can be approximately negative 88 degrees Celsius.

In FIG. 12D, the temperature sensor 1268 is located on an exterior surface of the expandable structure 144 of the end effector 122 (e.g., on a treatment side of the end effector 122 that is placed into contact with the target tissue during a treatment procedure). In an example, the temperature sensor 1268 located on the exterior surface of the expandable structure 144 can measure a temperature that can be indicative of an effectiveness of the treatment procedure. For instance, the temperature sensed by the temperature sensor 1268 can indicate when the target tissue has reached a desired temperature. In some implementations, the device 100 can include one or more components that are configured to provide a feedback loop for controlling the supply of cryogen to the end effector 122 based on the temperature sensed by the temperature sensor 1268. Although FIGS. 12A-12D show a single temperature sensor 1268 in different locations on the device 100, the device 100 can include one or more temperature sensors 1268 at one or more of the locations shown in FIGS. 12A-12D. As such, the device 100 can have a plurality of temperature sensors 1268 at a plurality of locations, including the locations shown and described above with respect to FIGS. 12A-12D.

As described above, in some examples of the device 100 shown in FIGS. 12A-12D, the temperature sensor 1268 can be used to measure, display, and/or control a temperature of surgical interest. For instance, in an implementation, the temperature sensor 1268 may be configured to sense the temperature of evaporating cryogen within the end effector 122. The temperature sensor 1268 may additionally or alternatively be configured to sense the temperature of a tissue of surgical interest.

The trigger 124 may also optionally include a servo mechanism configured to respond to a sensed temperature to modulate the flow of cryogen in order to control a desired surgical parameter. In particular, the device 100 may be configured to automatically adjust the flow rate of liquid cryogen in response to one or more of the following parameters: evaporator temperature, evaporator pressure, tissue temperature, evaporator exhaust gas temperature, or elapsed cryogen flow time. The flow rate may be adjusted in a continuous analog manner, and/or by an alternating on/off flow modulation.

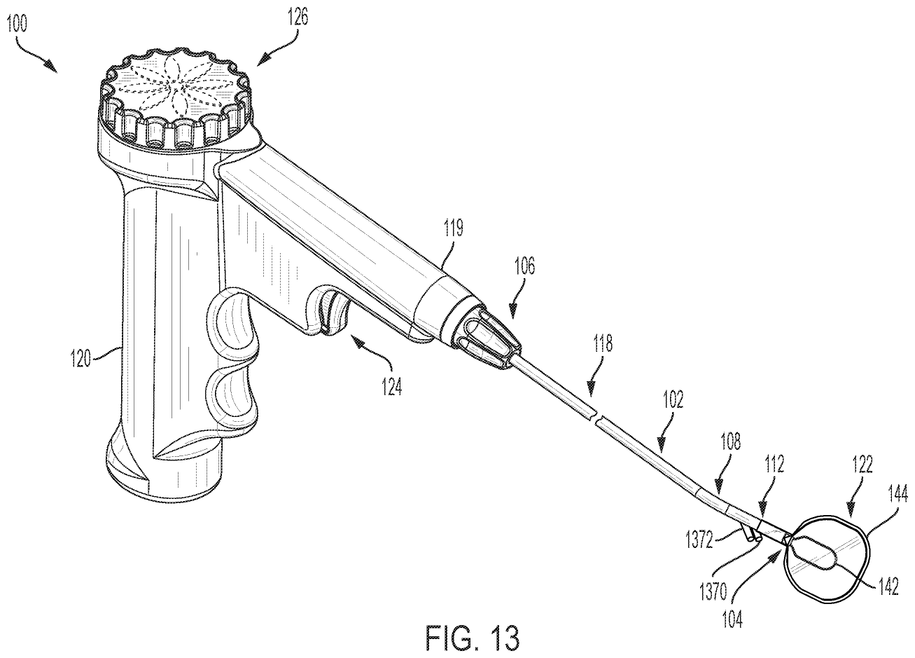

In addition to a temperature sensing capability, the device 100 may be configured with a camera and/or a light source disposed in the vicinity of the distal end 104 of probe shaft 102. The camera and/or the light source may be used, e.g., to identify nasal anatomical landmarks, and may be used to guide the placement of the end effector 122 against the lateral nasal wall for ablation of the function of a target posterior nasal nerve. FIG. 13 depicts the device 100 including a camera 1370 and a light source 1372 according to an example.

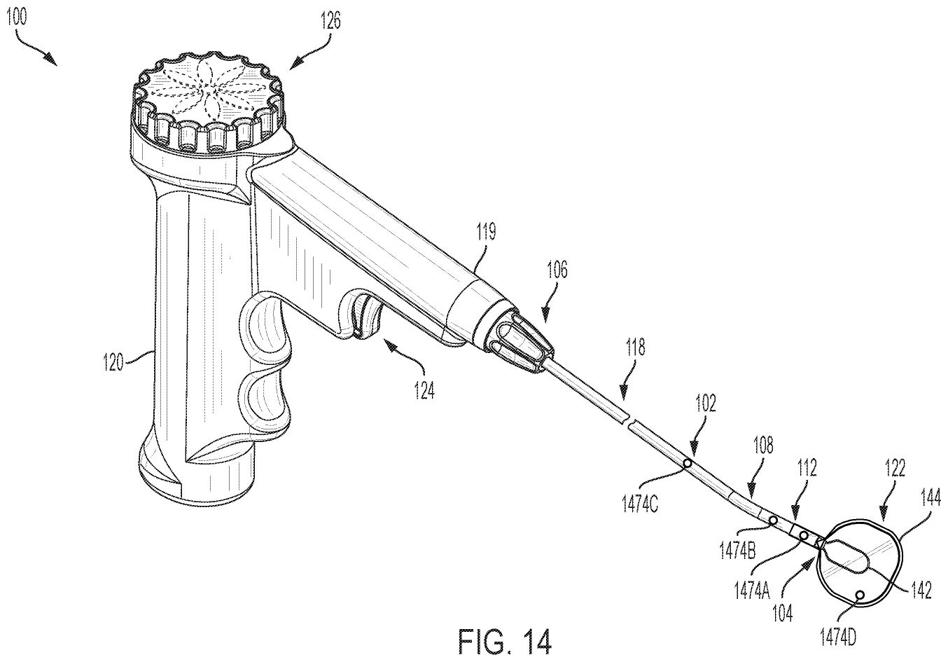

An ultrasonic or optical Doppler flow sensor may also be disposed in the vicinity of distal end 104 of probe shaft 102 and be used, e.g., to locate an artery associated with the target posterior nasal nerve, as a means for locating the target posterior nasal nerve. In one such example, the Doppler flow sensor includes an ultrasound detector. In another such example, the Doppler flow sensor includes an optical detector. In one example, the artery associated with the at least one nasal nerve includes an artery from a sphenopalatine branch. FIG. 14 depicts the device 100 including one or more Doppler flow sensors 1474A-1474D according to an example. In particular, the Doppler flow sensor 1474A and the Doppler flow sensor 1474B are located on the distal portion 112 of the probe shaft 102, the Doppler flow sensor 1474C is located on the proximal portion 118 of the probe shaft 102, and the Doppler flow sensor 1474D is located on the end effector 122.

Although FIG. 14 shows the device 100 having four Doppler flow sensors 1474A-1474D, the device 100 can have a lesser quantity or a greater quantity of Doppler flow sensors 1474A-1474D in other examples. Additionally, although FIG. 14 shows the Doppler flow sensors 1474A-1474D in particular locations on the device 100, the device 100 can include the one or more Doppler flow sensors 1474A-1474D in one or more alternative locations according to other examples.

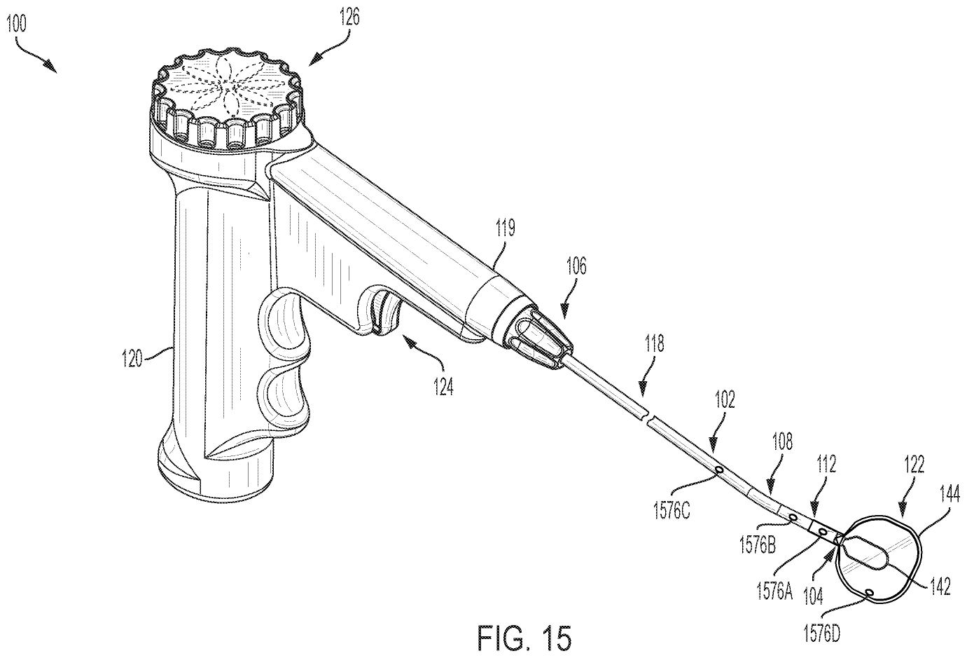

In addition, one or more electrodes may be disposed in the vicinity of the distal end 104 of probe shaft 102, which may be used for electrical stimulation or electrical blockade of the function of a target posterior nasal nerve using the observed physiological response to the stimulation or blockade to confirm correct surgical positioning of the end effector 122 prior to ablation and/or to confirm effectiveness of ablation by the determination of a change in the physiological response from before and after ablation. FIG. 15 depicts the device 100 including one or more electrodes 1576A-1576D according to an example. In particular, the electrode 1576A and the electrode 1576B are located on the distal portion 112 of the probe shaft 102, the electrode 1576C is located on the proximal portion 118 of the probe shaft 102, and the electrode 1576D is located on the end effector 122.

Although FIG. 15 shows the device 100 having four electrodes 1576A-1576D, the device 100 can have a lesser quantity or a greater quantity of electrodes 1576A-1576D in other examples. Additionally, although FIG. 15 shows the electrodes 1576A-1576D in particular locations on the device 100, the device 100 can include the one or more electrodes 1576A-1576D in one or more alternative locations according to other examples.

Any number of temperature sensing, endoscopic instruments, servo controlled cryogen control valves, ultrasonic or optical Doppler flow detection, and/or electrical nervous stimulation and blockade mechanisms may be optionally incorporated into the devices described herein.

In use, such a surgical probe may be used for treating a tissue region within a nasal cavity, generally comprising advancing a distal end of a surgical probe shaft through the nasal cavity and into proximity of the tissue region having a nasal nerve, introducing a cryogenic liquid into an expandable structure attached to the distal end of the probe shaft such that the expandable structure inflates from a deflated configuration into an expanded configuration against the tissue region, positioning a member relative to the tissue region, wherein the member is attached to the distal end of the probe shaft and extends within the expandable structure such that the member is unattached to an interior of the expandable structure, and wherein the member defines a flattened shape which is sized for placement against the tissue region proximate to the nasal nerve, and maintaining the member against the tissue region until the nasal nerve is cryogenically ablated.

Another example of the present disclosure is a cryo-surgical probe apparatus for ablation of a nasal nerve comprising a handle at the proximal end, a probe shaft with a spatula shaped cryo-ablation element mounted in vicinity of the distal end of the shaft, whereby the handle is configured for housing a cryogen source, and controlling the flow of the cryogen to the cryo-ablation element, and the geometric parameters of the probe shaft and cryo-ablation element are configured for cryo-ablation of nasal mucosa containing the nasal nerve according to the methods disclosed here within.

Another example of the present disclosure is a cryo-surgical probe apparatus for ablation of nasal mucosa comprising a handle at the proximal end, a probe shaft with a bullet shaped cryo-ablation element mounted in vicinity of the distal end of the shaft, whereby the handle is configured for housing a cryogen source, and controlling the flow of the cryogen to the cryo-ablation element, and the geometric parameters of the probe shaft and cryo-ablation element are configured for cryo-ablation of the nasal mucosa according to the methods disclosed here within.

Another example of the present disclosure is a cryo-surgical probe apparatus for ablation of a nasal nerve comprising a handle at the proximal end, a probe shaft with a bullet shaped cryo-ablation element mounted in vicinity of the distal end of the shaft, whereby the handle is configured for housing a cryogen source, and controlling the flow of the cryogen to the cryo-ablation element, wherein the probe shaft is configured with user operable deflectable distal segment, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve comprising a handle at the proximal end, a probe shaft with a cylindrically shaped cryo-ablation element mounted in vicinity of the distal end of the shaft, whereby the handle is configured for housing a cryogen source, and controlling the flow of the cryogen to the cryo-ablation element, wherein the cryo-ablation element includes a linear segmented cryo-ablation element, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve comprising a handle at the proximal end, a probe shaft with a cylindrically shaped cryo-ablation element mounted in vicinity of the distal end of the shaft, whereby the handle is configured for housing a cryogen source, and controlling the flow of the cryogen to the cryo-ablation element, wherein the cryo-ablation element includes a semi-circular cryo-ablation element, and the geometric parameters of the probe shaft and cryo-ablation element are configured for cryo-ablation of target tissue containing the nasal nerve according to the methods disclosed here within.

Another example of the present disclosure is a cryo-surgical probe apparatus for ablation of a nasal nerve comprising a handle at the proximal end, a probe shaft with a cylindrically shaped cryo-ablation element mounted in vicinity of the distal end of the shaft, whereby the handle is configured for housing a cryogen source, and controlling the flow of the cryogen to the cryo-ablation element, wherein the cryo-ablation element includes a spiraled cryo-ablation element, and the geometric parameters of the probe shaft and cryo-ablation element are configured for cryo-ablation of target nasal tissue containing the nasal nerve according to the methods disclosed here within.

Another example of the present disclosure is a cryo-surgical probe apparatus for ablation of a nasal nerve comprising a proximal end, a probe shaft with a cryo-ablation element comprising a balloon mounted in vicinity of the distal end of the shaft, whereby the proximal end is configured for receiving a cryogen from a cryogen source with the cryogen source comprising a means controlling the flow of the cryogen to the cryo-ablation element, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve comprising a handle at the proximal end, a probe shaft with a cylindrically shaped cryo-ablation element comprising a balloon mounted in vicinity of the distal end of the shaft, whereby the handle is configured for housing a cryogen source, and controlling the flow of the cryogen to the cryo-ablation element, and the geometric parameters of the probe shaft and cryo-ablation element are configured for cryo-ablation of target nasal tissue containing the nasal nerve according to the methods disclosed here within.

Another example of the present disclosure is a cryo-surgical probe apparatus for ablation of a nasal nerve comprising a handle at the proximal end, a probe shaft with a cylindrically shaped cryo-ablation element mounted comprising a balloon with two lateral chambers disposed in the vicinity of the distal end of the shaft, whereby the handle is configured for housing a cryogen source, and controlling the flow of the cryogen to the cryo-ablation element, wherein one chamber of the balloon is configured as a cryogen expansion chamber, and the second chamber is configured as a thermal insulation chamber, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve comprising a handle at the proximal end, a probe shaft with a “I” shaped cryo-ablation element comprising a balloon mounted in vicinity of the distal end of the shaft, whereby the handle is configured for housing a cryogen source, and controlling the flow of the cryogen to the cryo-ablation element, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve function comprising a handle at the proximal end, a probe shaft with a “J” shaped cryo-ablation element comprising a balloon mounted in vicinity of the distal end of the shaft, whereby the handle is configured for housing a cryogen source, and controlling the flow of the cryogen to the cryo-ablation element, and the geometric parameters of the probe shaft and cryo-ablation element are configured for cryo-ablation of the nasal nerve according to the methods disclosed here within.

Another example of the present disclosure is a cryo-surgical probe apparatus for ablation of a nasal nerve comprising a handle at the proximal end, a probe shaft with a cryo-ablation element mounted in vicinity of the distal end of the shaft, whereby the handle is configured for housing a cryogen source, and controlling the flow of the cryogen to the cryo-ablation element, wherein a suction means associated with the cryo-ablation element is configured for stabilizing the position of the cryo-ablation element against the target tissue, and the geometric parameters of the probe shaft and cryo-ablation element are configured for cryo-ablation of the nasal nerve according to the methods disclosed here within.

One aspect of the present disclosure is a method for cryo-surgical ablation of a nasal nerve comprising placing a film of oil or gel on the surface of a cryo-ablation element, then pressing the cryo-ablation element against the lateral wall of a nasal cavity adjacent to the nasal nerve, then ablating the nasal nerve with the cryo-ablation element, whereby the oil or gel prevents frozen nasal tissue from adhering to the cryo-ablation element.

Another aspect of the present disclosure is an electrosurgical probe apparatus for ablation of a nasal nerve comprising a handle at the proximal end, a probe shaft with a radiofrequency (RF) ablation element comprising at least one RF electrode mounted in the vicinity of the distal end of the shaft, an electrical connector in the vicinity of the handle configured to connect the RF ablation element to a source of radiofrequency energy, whereby the geometric parameters of the probe shaft and RF ablation element are configured for RF ablation of the nasal nerve according to the methods disclosed here within.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, and a fluid connector disposed in the vicinity of the handle to connect at least one fluid port associated with the RF ablation element with a source of pressurized liquid, whereby the geometric parameters of the probe shaft and RF ablation element are configured for RF ablation of the nasal nerve according to the methods disclosed here within.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according to the methods disclosed here within, wherein the RF ablation element includes a monopolar electrosurgical configuration comprising one or more electrodes.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according to the methods disclosed here within, wherein the RF ablation element includes a bi-polar electrosurgical configuration comprising two or more electrodes.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element, to a source of radiofrequency energy, whereby the geometric parameters of the probe shaft and RF ablation element are configured for RF ablation of the nasal nerve according to the methods disclosed here within, wherein the RF ablation element is disposed in the vicinity of the distal end of the shaft on a cylindrical, “J” shaped, “U” shaped or “T” shaped structure.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according to the methods disclosed here within, wherein the RF ablation element is configured in a lateral or radial arrangement.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according, to the methods disclosed here within, wherein the RF ablation element includes a circular array of domed electrodes disposed on a flat electrically insulative surface, with the domed electrodes optionally associated with a fluid irrigation port.

Another example of the present disclosure is an electrosurgical probe for ablation of the a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according to the methods disclosed here within, wherein the RF ablation element includes a linear array of domed electrodes disposed on a flat electrically-insulative surface, with the domed electrodes optionally associated with a fluid irrigation port, and a needle configured for injecting a liquid into a sub-mucosal space.

Another example of the present disclosure is an electrosurgical probe apparatus for ablation of a nasal nerve comprising a handle at the proximal end, a probe shaft with a RF ablation element comprising at least one RF electrode mounted in the vicinity of the distal end of the shaft, an electrical connector disposed in the vicinity of the handle configured to connect the RF ablation element to a source of radiofrequency energy, whereby the geometric parameters of the probe shaft and RF ablation element are configured for RF ablation of the nasal nerve according to the methods disclosed here within, wherein the RF ablation element includes at least one needle configured for interstitial RF ablation.

Another example of the present disclosure is an electrosurgical probe apparatus for ablation of a nasal nerve comprising a handle at the proximal end, a probe shaft comprising a distal and proximal end, and an integrated circuit comprising an RF generator disposed in the vicinity of the handle and an RF ablation element disposed in the vicinity of the distal end of the shaft, whereby the geometric parameters of the probe shaft and RF ablation element are configured for RF ablation of the nasal nerve according to the methods disclosed here within.

Yet another example of the present disclosure is an ultrasonic energy emitting probe apparatus for ablation of a nasal nerve comprising a handle at the proximal end, a probe shaft with an ultrasonic energy ablation element comprising at least one ultrasonic energy emitter mounted in the vicinity of the distal end of the shaft, an electrical connector in the vicinity of the handle configured to connect the ultrasonic energy emitter to an ultrasonic energy generator, whereby the geometric parameters of the probe shaft and ultrasonic energy emitter are configured for ultrasonic energy ablation of the nasal nerve according to the methods disclosed here within.

In another example of this disclosure is an ultrasonic energy emitting probe apparatus for ablation of a nasal nerve comprising a handle at the proximal end, a probe shaft with an ultrasonic energy ablation element comprising at least one ultrasonic energy emitter mounted in the vicinity of the distal end of the shaft, an electrical connector in the vicinity of the handle configured to connect the ultrasonic energy emitter to an ultrasonic energy generator; at least one fluid path in communication between at least one fluid connector in the vicinity of the handle and the ultrasonic energy emitter configured to cool the ultrasonic energy emitter during ultrasonic energy emission, whereby the geometric parameters of the probe shaft and ultrasonic energy emitter are configured for ultrasonic energy ablation of the nasal nerve according to the methods disclosed here within.

Methods of use of any of the devices described above are now provided. The posterior nasal nerves (PNN) include nerves that originate from the SPG and innervate the nasal mucosa on the posterior side of the nasal cavity. Ablating these nerves, as well as other nerves in the nasal cavity, leads to a decrease in or interruption of parasympathetic nerve signals that contribute to congestion and rhinorrhea in patients with chronic rhinitis (allergic or non-allergic). The devices and methods described herein are configured to be used for ablating one or more of these nasal nerves to reduce or eliminate rhinitis.

Generally, the devices described above may be used to ablate a nasal nerve of a nasal tissue region of a nasal cavity of a patient. One method for treating the nasal tissue region within a nasal cavity in proximity to the at least one nerve may include introducing a distal end of a probe shaft through the nasal cavity, wherein the distal end has an end effector with a first configuration having a low-profile which is shaped to manipulate tissue within the nasal cavity. The distal end may be positioned into proximity of the tissue region having the nasal nerve. Once suitably positioned, the distal end may be reconfigured from the first configuration to a second configuration, which is shaped to contact and follow the tissue region. The distal end may then be used to ablate the nasal nerve within the tissue region utilizing a number of different tissue treatment mechanisms, e.g., cryotherapy, as described herein.

In treating the tissue region in one specific variation, the distal end may be positioned specifically into proximity of the tissue region which is surrounded by the middle nasal turbinate, inferior nasal turbinate, and the lateral wall of the nasal cavity, forming a cul-de-sac and having the PNN. The distal end may be reconfigured to treat the tissue region accordingly.

Various configurations for the distal end may be utilized in treating the tissue region so long as the distal end is configured for placement within the narrowed confines of the nasal cavity and more specifically within the confines of the tissue region surrounding the middle nasal turbinate, inferior nasal turbinate, lateral nasal tissue wall, and inferior meatus. Other anatomical locations within the nasal cavity are alternatively or additionally treatable with the configurations described herein.

As described above, one example of a surgical probe configured for ablating a tissue region such as the nasal cavity includes a surgical probe apparatus having a surgical probe shaft comprising an elongated structure with a distal end and a proximal end, and an expandable structure attached to the distal end of the probe shaft, the expandable structure having a deflated configuration and an expanded configuration. A lumen may be defined through the shaft in fluid communication with an interior of the expandable structure. A member may be attached to the distal end and extend within the expandable structure which encloses the member such that the member is unattached to the interior of the expandable structure. Moreover, the member may define an atraumatic shape, which is sized for pressing against and manipulating through the expandable structure the nasal tissue region.

An example of utilizing such a structure in treating the tissue region may generally include advancing the distal end of the surgical probe shaft through the nasal cavity and into proximity of the target nasal tissue region having and introducing a cryogenic fluid into the expandable structure attached to the distal end of the probe shaft such that the expandable structure inflates from a deflated configuration into an expanded configuration against the target nasal tissue region.

A position of the member relative to the target nasal tissue region may be adjusted where the member is attached to the distal end of the probe shaft and extends within the expandable structure, which encloses the member such that the member is unattached to an interior of the expandable structure. The practitioner may apply a pressure against the distal end such that the member is pressed against the interior of the expandable structure which in turn is pressed against the target nasal tissue region, wherein the member defines an atraumatic shape which is sized for pressing against and manipulating the target nasal tissue region. The member may be maintained against the interior of the expandable structure and the target nasal tissue region until the target nasal tissue region is cryogenically ablated.

Any of the ablation devices herein can be used to ablate a single nerve branch or multiple nerve branches.

Another aspect of this disclosure is a method for treating rhinitis by ablating a nasal nerve. The method may include inserting the distal end of a surgical probe configured for cryo-neurolysis into a nostril of a patient. The surgical hand piece disposed on the proximal end of the probe shaft may include a liquid cryogen reservoir, as discussed above. The distal expandable structure may be positioned against the lateral nasal wall proximate to a target nasal nerve and then a flow of liquid cryogen to the expandable structure may be activated for a period of time sufficient to cryo-ablate a target area in the nose containing target nasal nerves.

The method may further involve the targeting of at least one additional posterior nasal nerve, either within the ipsilateral nasal cavity, or a posterior nasal nerve in a contralateral nasal cavity.

The method may include controlling the flow of the liquid cryogen into an evaporation chamber based on at least one predetermined parameter, which may include one or more of the following parameters: cryogenic liquid flow rate, cryogenic liquid flow elapsed time, cryogenic liquid evaporation pressure, cryogenic liquid evaporation temperature, cryogenic gas exhaust temperature, visual determination of tissue freezing, ultrasonic determination of tissue freezing, or the volume of cryogenic liquid supplied by the cryogenic liquid reservoir.

The method may include determining the location of the target nasal nerve, which may involve one or more of the following targeting techniques: endoscopic determination based on the nasal anatomical landmarks, electrical neuro-stimulation of the target nasal nerve while observing the physiological response to the stimulation, electrical neuro-blockade, while observing the physiological response to the blockade, or identification of the artery associated with the target nasal nerve using, e.g., ultrasonic or optical Doppler flow techniques.

Though the presently-disclosed devices and methods have primarily been discussed in the context of cryotherapy, the devices, systems, and methods described herein may have applicability with other ablative and non-ablative surgical techniques. For example, examples may include devices, systems, and methods that utilize heating/hyperthermia therapies. Examples utilizing heating/hyperthermia therapies may be similar in structure and steps as examples utilizing hypothermic therapies. Sources of heat for use with hyperthermia-based therapies may include RF energy, microwave energy, ultrasound energy, resistive heating, exothermic chemical reactions, combinations thereof and other heat sources known to those skilled in the art. Further, the disclosure may be applied as a standalone system or method, or as part of an integrated medical treatment system. It shall be understood that different aspects of the disclosure can be appreciated individually, collectively, or in combination with each other.

Further, though the presently-disclosed devices and methods have primarily been discussed in the context of ablating a least one nasal nerve associate with the lateral nasal wall of a nasal cavity of a patient, treatments may similarly be applied additionally or alternatively to the septal wall, roof of the nasal cavity, or other regions of the nasal cavity.

The methods described herein can be utilized effectively with any of the examples or variations of the devices and systems described above, as well as with other examples and variations not described explicitly in this document. The features of any of the devices or device components described in any of the examples herein can be used in any other suitable example of a device or device component.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

What is claimed is:

1. A device comprising:
a probe shaft having a distal end and a proximal end, wherein the probe shaft has a curved portion such that a longitudinal axis of a distal portion of the probe shaft has a non-zero angle with respect to a longitudinal axis of a proximal portion of the probe shaft, and wherein a flexibility of the proximal portion of the probe shaft is greater than a flexibility of the distal portion of the probe shaft;
a housing coupled to the proximal end of the probe shaft;
a handle coupled to the housing;
an end effector coupled to the distal end of the probe shaft, wherein the end effector defines an atraumatic surface when the distal end of the probe shaft is advanced through a nasal cavity of a patient and is positioned proximate to a nasal tissue region having at least one nasal nerve, and wherein the end effector is configured to transmit lateral pressure against the nasal tissue region; and
a trigger positioned in the handle, wherein activation of the trigger causes the end effector to ablate the at least one nasal nerve when the end effector is in contact against the nasal tissue region.

2. The device of claim 1, wherein the non-zero angle between the longitudinal axis of the distal portion of the probe shaft and the longitudinal axis of the proximal portion of the probe shaft is between about 15 degrees and about 25 degrees.

3. The device of any one of claims 1-2, wherein the curved portion of the probe shaft is positioned about 4 cm from the distal end of the end effector, and wherein the curved portion of the probe shaft causes a lateral deviation of the distal end of the end effector with respect to the longitudinal axis of the proximal portion of the probe shaft of about 1 cm.

4. The device of any one of claims 1-3, wherein the proximal end of the probe shaft extends into the housing.

5. The device of any one of claims 1-4, wherein the probe shaft is rotatable 180 degrees relative to the housing.

6. The device of any one of claims 1-5, wherein the distal portion of the probe shaft comprises a first material, and wherein the proximal portion of the probe shaft comprises a second material that is different than the first material.

7. The device of claim 6, wherein the first material comprises a polymer, and wherein the second material comprises stainless steel.

8. The device of any one of claims 1-7, wherein the proximal portion of the probe shaft comprises a first tube having a first diameter, and a second tube having a second diameter that is greater than the first diameter such that an air gap separates the first tube and the second tube.

9. The device of any one of claims 1-8, wherein the at least one nasal nerve comprises a posterior nasal nerve of a nasal branch of a vidian nerve.

10. The device of any one of claims 1-9, wherein the at least one nasal nerve comprises a parasympathetic nerve.

11. The device of any one of claims 1-10, wherein the end effector is configured to ablate the at least one nasal nerve using cryogenic fluid, RF energy, microwave energy, ultrasound energy, resistive heating, exothermic chemical reactions, or combinations thereof.

12. The device of any one of claims 1-11, further comprising:
a cryogenic fluid source positioned at least partially in the handle; and
a lumen disposed in the probe shaft and in fluid communication with the cryogenic fluid source.

13. The device of claim 12, wherein a height of the cryogenic fluid source is less than about 2 cm above the longitudinal axis of the proximal portion of the probe shaft.

14. The device of any one of claims 12-13, wherein the cryogenic fluid source comprises a canister that is removably positioned at least partially in the handle.

15. The device of any one of claims 12-14, wherein an angle between a longitudinal axis of the cryogenic fluid source and the longitudinal axis of the proximal portion of the probe shaft is between about 60 degrees and about 90 degrees, and preferably about 75 degrees.

16. The device of any one of claims 12-15, wherein the end effector comprises:
a planar member defining a flattened shape disposed at the distal end of the probe shaft, the planar member having an elongate structure with arcuate edges to define the atraumatic surface; and
an expandable structure surrounding the planar member and coupled to the distal end of the probe shaft, wherein the expandable structure is inflatable from a deflated configuration to an expanded configuration, and wherein an interior of the expandable structure is in fluid communication with the cryogenic fluid source.

17. The device of claim 16, wherein the expandable structure is configured to expand to a predetermined shape and size in the expanded configuration, and wherein the predetermined shape and size corresponds to a shape and size of the nasal tissue region.

18. The device of any one of claims 16-17, wherein the expandable structure is configured to transition from the deflated configuration to the expanded configuration upon evaporation of cryogenic fluid within the interior of the expandable structure.

19. The device of any one of claims 16-18, wherein the planar member comprises an elongate loop structure formed by a rigid wire that is configured to manipulate tissue in the nasal cavity.

20. The device of any one of claims 16-19, wherein the expandable structure has an expanded diameter between approximately 3 millimeters (mm) and 12 mm.

21. The device of any one of claims 16-20, wherein the planar member extends within the expandable structure such that it is unattached to an interior of the expandable structure.

22. The device of any one of claims 16-21, wherein the device is configured to cool an external surface of the expandable structure to between −20 degrees Celsius to −90 degrees Celsius for less than 120 seconds so as to controllably freeze the at least one nasal nerve at a depth less than 4 mm from a surface of the nasal tissue region so as to reduce at least one symptom of rhinitis of the patient.

23. A device comprising:
a probe shaft having a distal end and a proximal end, wherein the probe shaft has a curved portion positioned between a distal portion of the probe shaft and a proximal portion of the probe shaft such that a longitudinal axis of a distal portion of the probe shaft has a non-zero angle with respect to a longitudinal axis of a proximal portion of the probe shaft, and wherein the proximal portion of the probe shaft comprises a first tube having a first diameter and a second tube having a second diameter that is greater than the first diameter such that an air gap separates the first tube and the second tube;
a housing coupled to the proximal end of the probe shaft;
a handle coupled to the housing;
an end effector coupled to the distal end of the probe shaft, wherein the end effector defines an atraumatic surface when the distal end of the probe shaft is advanced through a nasal cavity of a patient and is positioned proximate to a nasal tissue region having at least one nasal nerve, and wherein the end effector is configured to transmit lateral pressure against the nasal tissue region; and
a trigger positioned in the handle, wherein activation of the trigger causes the end effector to ablate the at least one nasal nerve when the end effector is in contact against the nasal tissue region.

24. A method for treating a nasal tissue region of a nasal cavity of a patient, the method comprising:
introducing a distal end of a probe shaft through the nasal cavity, wherein the distal end of the probe shaft has an end effector with a first configuration having a low-profile which is shaped to manipulate tissue within the nasal cavity, wherein the probe shaft has a curved portion such that a longitudinal axis of a distal portion of the probe shaft has a non-zero angle with respect to a longitudinal axis of a proximal portion of the probe shaft, and wherein a stiffness of the proximal portion of the probe shaft is greater than a stiffness of the distal portion of the probe shaft;
reconfiguring the end effector from the first configuration to a second configuration in which the end effector is shaped to contact and follow a contour of the nasal tissue region; and
ablating, via the end effector, at least one nasal nerve of the nasal tissue region until symptoms of rhinitis are reduced.

25. The method of claim 24, the at least one nasal nerve of the nasal tissue region is associated with a middle or inferior nasal turbinate.

26. The method of any one of claims 24-25, wherein the at least one nasal nerve comprises a posterior nasal nerve of a nasal branch of a vidian nerve.

27. The method of any one of claims 24-25, wherein the at least one nasal nerve comprises a parasympathetic nerve.

28. The method of any one of claims 24-27, wherein the distal end of the probe shaft is advanced through the nasal cavity of the patient and in proximity of a sphenopalatine foramen.
